(19) United States
(12) Patent Application Publication
(10) Pub. No.: US 2001/0012620 A1
(43) Pub. Date: Aug. 9, 2001

(54) Method of reducing cell proliferation by inducing apoptosis in differentially selected cell subpopulations

(76) Inventor: Ivan N. Rich, Columbia, SC (US)

Correspondence Address: WOMBLE CARLYLE SANDRIDGE & RICE, P.O. Box 725388, Atlanta, GA, 31139-9388, US

(21) Appl. No.: 09/777,176

(22) Filed: Feb. 5, 2001

Related U.S. Application Data
(63) Continuation-in-part of application No. 09/325,444, filed on Jun. 3, 1999.
(60) Provisional application No. 60/087,864, filed on Jun. 3, 1998.
(60) Provisional application No. 60/252,882, filed on Nov. 22, 2000.

(51) Int. Cl. G01N 33/53; A61K 31/4965

(52) U.S. Cl. 435/7.1; 514/255.06

(57) ABSTRACT

The present invention relates to simultaneously combining intracellular pH (pH_{I}) measurements, membrane antigen expression, and cell cycle parameters to analyze proliferating cell populations. The present invention also relates to inducing a state of apoptosis in proliferating cells by decreasing the pHᵢ by inhibiting the cellular NHE. The present invention, therefore, provides a method of identifying a subpopulation of cell in the S-phase of the cell cycle, as a function of the pHᵢ of the cell population, and selectively isolating this subpopulation of cells. The present invention also provides a method of selectively isolating a subpopulation of cells having a cell surface antigen. The present invention further provides methods of reducing cellular proliferation by inducing cellular apoptosis in a subpopulation of proliferating cells by contacting the cells with an NHE inhibitor, thereby reducing the pH₁ and inducing a state of apoptosis.

### PRIORITY CLAIM

This application is a continuation-in-part of non-provisional application Ser. No. 09/325,444, filed Jun. 3, 1999 and which is hereby incorporated by reference in its entirety. The applicant claims the benefit of the filing dates of U.S. provisional applications, Ser. No. 60/087,864, filed Jun. 3, 1998, and Ser. No. 60/252,882, filed Nov. 22, 2000, the contents of each of which are also hereby incorporated by reference in their entireties.

### FIELD OF THE INVENTION

The present invention relates generally to methods of detecting subpopulations of cells and inhibiting the proliferation thereof. In particular, the present invention relates to methods of detecting subpopulations of cells having a high internal pH and methods of inducing apoptosis therein by inhibiting the Na⁺/H⁺ exchanger.

### BACKGROUND

The lympho-hematopoietic system is a structured, hierarchical and precisely regulated biological system capable of maintaining, within a finite range, the continuous production of at least eight different cell types, each with a specific function. It is estimated that in 1998, malignancies of the blood-forming system, including lymphoma, multiple myeloma and leukemia, constituted about 8.6% of the new cancer cases reported in the U.S. Leukemia represented the 5^{th} and 6^{th} cause of cancer-related deaths in men and women respectively in 1998. New methods of diagnosis and treatment, therefore, are still required to increase the survival rate caused by lympho-hematopoietic malignancies.

Cells are equipped with several exchangers to regulate the intracellular pH (pHᵢ) and counteract acidification either by the efflux of H⁺ ions or the influx of HCO₃⁻ions. The Na⁺-dependent and independent Cl⁻/HCO₃⁻exchangers and an ATP-dependent H⁺ pump are important if the cell becomes too acidic or too alkaline. See Fleigel et al., Biochem. J. 296, 273-285 (1993); Wakabayashi et al., Physiol. Rev. 77, 51-74 (1997). The Na⁺-dependent Cl⁻/HCO₃⁻ antiporter exchanges Na⁺ and HCO₃⁻ ions for Cl⁻ ions, causing an increase in cytosolic pHᵢ. The Na⁺-independent Cl⁻/HCO₃⁻ exchanger reduces the pHᵢ in cells having an alkali overload. The primary regulator of pHᵢ, however, is the Na⁺/H⁺ exchanger (NHE), of which there are six known isoforms. See Orlowski et al., J. Biol. Chem. 272, 22373-22376 (1997).

The ubiquitous, amiloride-sensitive, Na⁺/H⁺ exchanger isoform 1 (NHE-1) represents one of the primary mechanisms by which cells regulate intracellular pH (pH₁) and cell volume. See Fleigel et al., Biochem. J. 296, 273-285 (1993); Wakabayashi et al., Physiol. Rev. 77, 51-74 (1997). The human NHE-1 is an 815 amino acid membrane protein transporter produced from the 70 kb long APNH gene on chromosome 1p35-36.1 (Ludt et al., J. Memb. Biol. 134, 143-153 (1993)). The NHE-1 protein has 12 membrane-spanning segments. The extracellular region contains a highly conserved Na⁺/H⁺ binding site and an amiloride-binding site. The cytoplasmic domain of NHE-1 includes the pH sensor and maintenance sites (Wakabayashi et al., Physiol. Rev. 77, 51-74 (1997)). It further includes regions that activate the exchanger when growth factors, mitogens or non-mitogenic signals act on the cell (Wakabayashi et al., Physiol. Rev. 77, 51-74 (1997)). For this reason, the NHE-1 has also been called a “growth factor-activatable” exchanger.

Activation of NHE-1 results in the 1:1 stoichiometric efflux of H⁺ and influx of Na⁺ ions, and a concomitant increase in the pH₁. All of the growth factors tested have been shown to increase the pHᵢ with an associated activation of cell stimulation and proliferation (Fleigel et al., Biochem. J. 296, 273-285 (1993)). For example, using the interleukin-3 (IL-3)-dependent stem cell line, FDCP-mix, Whetton et al., in Biochem J. 256, 585-592 (1988) showed that IL-3 activated NHE-1, and that the resulting intracellular alkalinisation was a signal for proliferation of these cells. Granulocyte-macrophage colony-stimulating factor (GM-CSF) that stimulated granulocyte and macrophage proliferation also activated the NHE. See Valance et al., Biochem. J. 265, 359-364 (1990). Removal of growth factors such as IL-2 from IL-2-dependent cytotoxic T cells decreased the pHᵢ and induced apoptosis (Eastman, Li J. J. Biol. Chem. 270. 3203-3211 (1995); Rebollo et al. Exp. Cell Res. 218, 581-585 (1995)).

Cell-cell interactions can also activate the NHE-1. Many members of the integrin family can activate the exchanger (for example, see Schwartz et al., Exp. Cell Res. 195, 533-535 (1991)) and NHE-1 is involved in cell-cell interactions of normal murine hematopoietic cells (Rich et al., J. Cell Physiol. 177, 109-122 (1998)). When murine bone marrow cells are brought together at high cell density by centrifugation, hematopoietic stem and progenitor cells can be stimulated to produce colonies in vitro in the absence of growth factors. The stimulation is due to the interaction of the α₄ integrin subunit with fibronectin, causing activation of NHE-1, an increase in pHᵢ and increased hematopoietic colony formation (Rich et al., J. Cell Physiol. 177, 109-122 (1998)).

In cancers and hematopoietic malignancies in particular, only a small subpopulation of the cells are dividing. For example, in chronic myelogenous leukemia (CML) 5.8% of cells in peripheral blood and 12.2% of bone marrow cells are in the S+G2+M proliferative phases of the cell cycle. For acute myelogenous leukemia (AML) the corresponding amounts are 2.4% and 7.1% respectively and for acute lymphoblastic leukemia (ALL) 1.9% and 7.1% respectively. Thus, even in a patient with a high proportion of blasts, only a small percentage of the total cell population would be proliferating. It is, therefore, important to differentiate between the proliferating and non-proliferating leukemic cell population, since it is the former that is producing the latter.

Amiloride is a potassium-sparing diuretic typically used to treat hypokalemia, the management of edema and used as an adjunct in hypertension. Many amiloride derivatives and analogs have been produced with variable potency and specificity (Kleyman & Cragoe. J. Membr. Biol. 105, 1-21 (1988)) and have been used in various applications with hematopoietic cells. In different leukemic cell lines, when the NHE was inhibited by amiloride analogues, acidification of the cells occurred with induction of apoptosis (Perez-Sala et al., J. Biol. Chem. 270, 6235-6242 (1995); Chen et al., J. Cell Sci. 110, 379-387 (1997); Tsao & Lei, J. Immunol. 157, 1107-1116 (1996)). Cells maintaining a high rate of proliferation have a sustained increase in pHᵢ relative to normal cells because of activation of the sodium/hydrogen exchanger, and have increased sensitivity to NHE inhibitors.

It would be advantageous, therefore, to have methods that could be used to identify potential cell subpopulations as targets for NHE inhibitors, and to evaluate the use of such inhibitors as effective therapeutic agents against these identified subpopulations. It would also be advantageous to have new methods for reducing cell proliferation and/or inducing apoptosis in leukemic and other cancer cells.

These and other objectives and advantages of the invention will become fully apparent from the description and claims that follow or may be learned by the practice of the invention.

### SUMMARY OF THE INVENTION

Briefly described, the present invention relates to the determination of the cell cycle status of a population of human or animal cells, the percentage of cells in the S-phase of the cell cycle correlating to the average cellular internal pH (pHᵢ) of the cell population. The higher the pHᵢ value, the greater the percentage of cells in the S-phase. The present invention simultaneously combines pHᵢ measurements, the detection of membrane antigens and cell cycle parameters, to analyze proliferating cell populations. The present invention also relates to inducing a state of apoptosis in proliferating cell subpopulations by decreasing the pHᵢ by inhibiting the cellular NHE.

The present invention addresses the need for methods of selectively identifying populations of proliferating cells having increased pHᵢ values and subpopulations of cells therein. The present invention further addresses the need to inhibit the proliferation of those cells distinguished by having an increased pHᵢ. One aspect of the present invention is methods useful for the identification and isolation of subpopulations of proliferating cancerous cells, and the optimization of NHE inhibitors for inhibiting the proliferation of the cells by inducing apoptosis therein. The methods of the present invention are especially useful for inhibiting the proliferation of leukemic cells, but can also be used to kill or inhibit the proliferation of other cancer cells.

The present invention addresses these needs by providing methods for determining the cell cycle status of a population of human or animal cells, selecting a subpopulation of proliferating cells defined by the cellular complement of cell surface antigens, and inducing apoptosis and reducing proliferation in the proliferating cells by inhibiting the cellular NHE.

The methods of the present invention may be used for inducing apoptosis in a subpopulation of cells by inhibiting the NHE of isolated cells that, once treated, can be returned to the patient. The methods can also be used to optimize therapeutic doses of an NHE inhibitor before administering the inhibitor to a patient to induce apoptosis in cell subpopulations in vivo.

The present invention also addresses the need for simple methods of identifying subpopulations of proliferating cells by supplying kits containing pH and cell surface antigen indicators, and instructions for their use according to the present invention. The present invention further addresses the need for simple methods of inhibiting the proliferation of subpopulations of cells by supplying kits containing pH and cell surface antigen indicators and at least one NHE inhibitor, and instructions for their use according to the present invention.

Additional objects and aspects of the present invention will become more apparent upon review of the detailed description set forth below when taken in conjunction with the accompanying figures, which are briefly described as follows.

### BRIEF DESCRIPTION OF THE FIGURES

FIGS. 1A and 1B illustrate the phenotypic analysis of human acute lymphocytic leukemic (ALL) peripheral blood cells. FIG. 1A shows the analysis prior to, and FIG. 1B shows the analysis after, 3 months of growth and expansion as nodules in NOD-SCID mice. Also shown in FIG. 1B is the phenotypic characterization of mouse and human CD45⁺ cells derived from the nodules.

FIG. 2 shows the comparison of pHᵢ values from normal peripheral blood and bone marrow with those from 7 different human leukemic cell lines. Values represent the mean±SEM of at least 4 experiments for each cell line tested. All cell lines exhibited a statistically higher (p<0.01) pHᵢ than normal cells.

FIG. 3 illustrates a dot plot of normal peripheral blood, wherein the event clusters have been divided into specific populations. The “lymphocyte” population that contains CD34⁺ cells was divided into three regions (R2, R3 and R4). The fluorescent ratio histograms and mean values for each of the regions are shown for regions R6 to R10 representing the dot plot regions R1 to R5 respectively.

FIG. 4 illustrates box and whisker plots for normal peripheral blood and bone marrow (as shown in FIG. 1) and primary leukemic patient samples. Lowest and highest boundaries of the box indicate the 25^{th} and 75^{th} percentiles respectively; the whiskers above and below the box designate the 95^{th} and 5^{th} percentiles respectively; the solid line within the box represents the median value while the dotted line is the mean value; dots above or below the box indicate outliers. Acute lymphoblastic leukemia (ALL, n=5), acute myelogenous leukemic (AML, n=5), chronic lymphocytic leukemia (CLL, n=5), chronic myelogenous leukemia (CML, n=6), pre-B-acute lymphoblastic leukemia (pre-B-ALL, n=5).

FIG. 5 illustrates a pHᵢ analysis from a patient with CML in chronic phase with only 2.5% blasts. The pHᵢ of R1 (dot plot) corresponds to R4 in the histogram. Region R2 corresponds to R5, and R3 corresponds to R6. In the absence of any regions, the pHᵢ was 7.31.

FIG. 6 illustrates the correlation between pHᵢ and cell cycle. Samples from different normal and leukemic cell suspensions were divided into two aliquots, one for pHᵢ and the other for cell cycle measurements. The graph shows the number of values for the individual samples, the linear regression (r²=0.911) and the 95% confidence limits of the regression line.

FIGS. 7A-C illustrate the effect of HMA induction of apoptosis upon RNA integrity in KG-1A leukemic cells (FIG. 7A), RS411 and SUP. B8 leukemic cells (FIG. 7B) and MOLT-4 leukemic cells (FIG. 7C).

FIGS. 8A-D illustrate the effect of HMA on the pHᵢ and apoptosis of normal peripheral blood cells. Not shown on the graphs are the pHᵢ and apoptosis control time points in the absence of HMA. These are as follows: (A) Fluorescence ratio imaging microscopy (FRIM): 30 min=6.95±0.02, 1 h=7.00±0.015, 3 h=7.08±0.02, 5 h= 7.13±0.02; (B) Flow cytometry: 30 min=7.01±0.03, 1 h=6.98±0.04, 3 h=7.04±0.04, 5 h=7.09±0.05; (C) Annexin-V-FITC: 30 min=1.53±0.09 1 h=1.32±0.15, 3 h=1.38±0.04, 5 h=2.6±0.07; and. (D) TUNEL: 30 min=0.89±0.31, 1 h=0.96± 0.16, 3 h=0.60±0.29, 5 h=1.8±0.48. Results represent mean±SEM of 3 experiments.

FIGS. 9A and 9B illustrate the effect of HMA on pHᵢ and apoptosis of KG-1a leukemic cells. Measurement of pH₁ (FIG. 9A) was performed using flow cytometry. Apoptosis (FIG. 9B) was detected by annexin-V-FITC. Results represent the mean±SEM of 3 experiments.

FIGS. 10A and 10B illustrate the effect of HMA on pHᵢ and apoptosis of primary acute lymphoblastic leukemic cells. FIG. 10A illustrates the measurement of pHᵢ by FRIM and flow cytometry. FIG. 10B illustrates the estimation of apoptosis by annexin-V-FITC and TUNEL assay. Results represent mean±SEM of 3 experiments.

FIG. 11 illustrates flow cytometric histogram profiles of a representative ALL sample incubated in the absence or presence of HMA, followed by measurement of apoptosis by the TUNEL method.

### DETAILED DESCRIPTION OF THE PREFERRED EMBODIMENTS

A full and enabling disclosure of the present invention, including the best mode known to the inventor of carrying out the invention is set forth more particularly in the remainder of the specification, including reference to the Examples. This description is made for the purpose of illustrating the general principles of the invention and should not be taken in the limiting sense.

The methods of the present invention can be used to analyze populations of cells and differentially distinguish proliferating cells from non-proliferating cells, wherein the proliferating cells have an increased intracellular pH. The increased pHᵢ of proliferating cells renders the cells susceptible to inhibitors of the NHE. Inhibition of the NHE and the consequent decrease in the pHᵢ induces apoptosis and the death of the treated cells. The methods of the present invention further allow for distinguishing subpopulations of cells, within a population of proliferating cells, that may differ in response to an NHE inhibitors. This allows for the optimization of the administered NHE inhibitors to achieve maximum efficacy against a tumor or cancer.

The methods of the present invention combine pHᵢ measurements, membrane antigen expression, and cell cycle parameters, to specifically analyze subpopulations of proliferating cells. The present invention also provides methods to identify a proliferating cell population, including, for example, a leukemic cell population, by comparing the pHᵢ and cell cycle status of non-proliferating and normal resting cell populations.

The methods of the present invention may be applied to any population of cells, including cells isolated from tissues and solid tumors. The methods of the present invention are especially useful when applied to populations of hematopoietic cells including peripheral blood cells and bone marrow cells, and particularly to identify subpopulations of proliferating leukemic cells on the basis of a cell's complement of cell surface antigens. The differentially distinguished cell subpopulations may be isolated and treated with NHE inhibitors. Non-proliferating or normal cells may also be treated, but will be less sensitive to the inhibitor.

The methods of the present invention can also be used to distinguish subpopulations of cells that may differ in the response to NHE inhibitors. The methods may be used to optimize the inhibitors to achieve maximum efficacy against a subpopulation of proliferating cells. An optimized NHE inhibitor dose, determined from an isolated small sample of the cell population of a patient, may be administered to the proliferating cells in vivo, wherein the optimized dose may be administered systemically to the human or animal patient having the proliferating subpopulation of cells.

In methods of the present invention, a harvested cell population is contacted with a pH indicator. The cells may be any hematopoietic cells or obtained from a solid tumor or tissue. A solid tumor or tissue can be disrupted mechanically, enzymatically or by any combination thereof, to give a suspension of the cells in a liquid medium. The proportion of cells in the S-phase of the cell cycle increases with an increase in the pHᵢ, thereby allowing proliferating cells to be isolated by flow cytometry and cell sorting on the basis of their pHᵢ value. Proliferating cells may be further characterized into differentially distinguishable subpopulations (simultaneously with the determination of the pHᵢ if so desired) by adding indicators specific for cell surface antigens to the cells.

The present invention also provides methods for inhibiting cell proliferation by inhibiting the NHE of cells having an increased pHᵢ. This results in a decrease in the intracellular pH, the induction of the apoptotic process, and cell death.

### Definitions

The term “cell cycle” as used herein refers to the cycle of stages in the replication of a eukoryotic cell. The cycle comprises the four stages G1, S, G2 and M, wherein the S phase is that portion of the cycle wherein the nucleic acid of the cell is replicated. Thus, a cell identified as being in the S-phase of the cell cycle is also identified as being a proliferating cell.

The terms “pHᵢ” or “intracellular pH” as used herein refers to the internal pH of a cell, as opposed to the pH of the external milieu of the cell.

The term “fluorescent” as used herein refers to the excitation of a molecule by radiation including, but not limited to, ultra-violet light, visible light, infra-red light, laser emissions thereof and any single or multiple wavelengths thereof that will result in the emission of radiation by the excited molecule at a wavelength different from that of the exciting irradiation.

The term “pH indicator” as used herein refers to any compound or combination of compounds or derivatives thereof, that has one detectable property when at a first pH, and a second detectable property when at a second pH. The pH indicator may display a shift in a property, the content of which is a function of the pH, or the ratio of properties is a function of pH. The “pH indicator” may be, but is not limited to, a fluorescent pH indicator. Suitable pH indicators include, for example, 1,4-diacetoxy-2,3-dicyanobenzene (ADB), 4-methylumbelliferone (4-MU), 2′,7′-bis-carboxyethyl-5(6)-carboxyfluorescein (BCECF) and carboxy-SemiNapthoRhoda-Fluor-1 acetoxymethyl ester, acetate (carboxy SNARF-1 AM). A fluorescent pH indicator may be detected, for example, by a flow cytometer or fluorescence ratio imaging microscope. The “pH indicator” may be physiologically acceptable to human or animal cells.

The term “animal” as used herein refers to any vertebrate animal other than a human having a population of cells wherein at least one subpopulation of the cells may be proliferating or induced to proliferate.

The term “flow cytometer” as used herein refers to any device that will irradiate a particle suspended in a fluid medium with light at a first wavelength, and is capable of detecting a light at the same or a different wavelength, wherein the detected light indicates the presence of a cell or an indicator thereon. The “flow cytometer” may be coupled to a cell sorter that is capable of isolating the particle or cell from other particles or cells not emitting the second light.

The term “primary cell” refers to cells obtained directly from a human or animal adult or fetal tissue, including blood. The “primary cells” or “cell lines” may also be derived from a solid tumor or tissue, that may or may not include a hematopoietic cell population, and can be suspended in a support medium. The primary cells may comprise a primary cell line.

The term “cell line” refers to cells that are harvested from a human or animal adult or fetal tissue, including blood and cultured in vitro, including primary cell lines, finite cell lines, continuous cell lines, and transformed cell lines.

The term “cell” or “cells” as used herein refers to any cell population of a solid or non-solid tissue including, but not limited to, a peripheral blood cell population, bone marrow cell population, a leukemic cell line population and a primary leukemic cell line population or a blood stem cell population. The cells may be hematopoietic cells, including bone marrow, umbilical cord blood, fetal liver cells, yolk sac and differentiating embryonic stem cells or differentiating primordial germ cells or embryonic germ cells. The cells may be a primary cell line population including, but not limited to, a leukemic cell line. Examples of leukemic cell lines include, but are not limited to, an acute lymphocytic leukemia, an acute myeloid leukemia, a chronic lymphocytic leukemia, a chronic myeloid leukemia and a pre-B acute lymphocytic leukemia. Such cell lines include, but are not limited to, acute myelogenous leukemia, acute T-cell leukemia, acute lymphoblastic leukemia, chronic myeloid leukemia, acute monocytic leukemia and B-cell leukemia.

The term “disrupt” as used herein refers to destroying the structural integrity of a tissue or organ of a human or animal by applying mechanical, enzymatic or chemical methods, or any combination thereof, that will release cells from supporting or confining connective tissue.

The term “tissue” as used herein refers to a group or collection of similar cells and their intercellular matrix that act together in the performance of a particular function, the primary tissues are epithelial, connective (including blood), skeletal, muscular, glandular and nervous.

The term “enzyme capable of disrupting” as used herein refers to any enzyme that can partially or totally digest connective tissue or intercellular matrix that confines cells to a tissue. Such enzymes include, but are not limited to, proteases such as, for example, collagenase, elastase, trypsin, chymotrypsin, enterokinase, proteinase K and the like, lipases, and glycosidases that alone or in combination will sufficiently destroy the integrity of the intracellular matrix that individual cells or cell clumps may be released into a suspending medium.

The term “suspension” as used herein refers to cells, or small clumps of cells, released from a tissue or organ and surrounded by a fluid.

The term “pharmaceutically acceptable” as used herein refers to a compound or combination of compounds that, while biologically active, will not damage the physiology of the recipient human or animal to the extent that the viability of the recipient human or animal is reduced. Preferably, the administered compound or combination of compounds will elicit, at most, a temporary detrimental effect on the health of the recipient human or animal.

The terms “cell surface antigen” and “cell surface marker” as used herein may be any antigenic structure on the surface of a cell. The cell surface antigen may be, but is not limited to, a tumor associated antigen, a growth factor receptor, a viral-encoded surface-expressed antigen, an antigen encoded by an oncogene product, a surface epitope, a membrane protein which mediates a classical or atypical multi-drug resistance, an antigen which mediates a tumorigenic phenotype, an antigen which mediates a metastatic phenotype, an antigen which suppresses a tumorigenic phenotype, an antigen which suppresses a metastatic phenotype, an antigen which is recognized by a specific immunological effector cell such as a T-cell, and an antigen that is recognized by a non-specific immunological effector cell such as a macrophage cell or a natural killer cell. Examples of “cell surface antigens” within the scope of the present invention include, but are not limited to, CD5, CD30, CD34, CD45RO, CDw65, CD90 (Thy-1) antigen, CD117, CD38, and HLA-DR, AC133 defining a subset of CD34⁺ cells, CD19, CD20, CD24, CD10, CD13, CD33 and HLA-DR. Also contemplated to be within the scope of the present invention are cell surface molecules, including carbohydrates, proteins, lipoproteins or any other molecules or combinations thereof, that may be detected by selectively binding to a ligand or labeled molecule by methods such as, but not limited to, flow cytometry, FRIM, fluoresence microscopy and immunohistochemistry.

The term “cell surface indicator” as used herein refers to a compound or a plurality of compounds that will bind to a cell surface antigen directly or indirectly, and thereby selectively indicate the presence of the cell surface antigen. Suitable “cell surface indicators” include, but are not limited to, cell surface antigen-specific monoclonal or polyclonal antibodies, or derivatives or combinations thereof, and which may be directly or indirectly linked to a signaling moiety. The “cell surface indicator” may be a ligand that can bind to the cell surface antigen, wherein the ligand may be a protein, peptide, carbohydrate, lipid or nucleic acid that is directly or indirectly linked to a signaling moiety.

The term “apoptosis” as used herein refers to the onset and progression of morphological alterations, including cell shrinkage, membrane blebbing, chromatin condensation and fragmentation exhibited by dying cells. A change in a cell, or the environment of the cell, may trigger the progressive apoptotic degradation of the cellular components including, for example, the nucleic acids, such as DNA, messenger RNA, transfer RNA, ribosomal RNA, and proteins. The term “apoptosis” also refers to “programmed cell death” which is the induction of specific genetic pathways leading to cell death.

The term “quiescent” refers to cells are not actively proliferating by means of the mitotic cell cycle. Quiescent cells (which include cells in which quiescence has been induced as well as those cells which are naturally quiescent, such as certain fully differentiated cells) are generally regarded as not being in any of the four phases G1, S, G2 and M of the cell cycle; they are usually described as being in a Gₒ state, so as to indicate that they would not normally progress through the cycle. Cultured cells can be induced to enter the quiescent state by various methods including chemical treatments, nutrient deprivation, growth inhibition or manipulation of gene expression.

The terms “Na⁺/H⁺ exchanger”, “NHE” or “NHE-1” as used herein refer to an amiloride-sensitive, sodium/hydrogen exchanger that represents one of the primary mechanisms by which cells regulate intracellular pH (pHᵢ) and cell volume. The sodium/hydrogen exchanger includes isoforms including, but not limited to, the human NHE-1 isoform which is an 815 amino acid membrane protein transporter produced from the 70 kb long APNH gene on chromosome 1p35-36.1. The extracellular region of NHE-1 contains a highly conserved Na⁺/H⁺ binding site as well as an amiloride-binding site. It is contemplated that the methods of the present invention for inducing apoptosis by increasing intracellular pH levels, may be applied to any isoform of the NHE, including, but not limited to, NHE-1, and which may be inhibited by an NHE inhibitor.

The terms “Na⁺/H⁺ exchanger inhibitor”, “NHE inhibitor” and “NHE-1 inhibitor” as used herein include any compound or combination of compounds capable of totally or partially inhibiting the ability of the Na⁺/H⁺ exchanger to remove hydrogen ions from the interior of a cell. Suitable NHE inhibitors include, but are not limited to, amiloride (3,5-Diamino-N-(aminoiminomethyl)-6-chloropyrazinecarboxamide; N-amidino-3,5-diamino-6-chloropyrazine carboxamide), and amiloride derivatives such as, but not limited to, 5-(N,N-hexamethylene)-amiloride (HMA), 5-(N,N-ethyl-N-isopropyl)-amiloride (EIPA), and 5-N-methyl-N-isobutyl)-amiloride (MIA), simvastatin and phenamil, and non-amilorides such as, but not limited to, (2-methyl-5-(methylsulfonyl)-4-pyrrolobenzoyl)-guanidine (EMD), (3-methylsulfonyl-4-piperidinobenzoyl)guanidine methanesulfonate) (Hoe 694), CARIPORIDE™ (Hoe 642), cimetidine, clonidine, hormaline.

### Abbreviations

Abbreviations used in the present specification include the following: pHᵢ: intracellular pH; NHE, Na⁺/H⁺ exchanger; NHE-1, Na⁺/H⁺ exchanger-isoform 1; IL, interleukin; ALL, acute lymphocytic leukemia; AML, acute myelogenous leukemia; CLL, chronic lymphocytic leukemia; CML, chronic myelogenous leukemia; pre-B-ALL, preB-acute lymphocytic leukemia; NOD-SCID, Non-Obese Diabetic, Severe Combined Immunodeficient; HMA, 5-(N,N-hexamethylene)-amiloride; TUNEL, terminal deoxynucleotidyl transferase (Tdt)-mediated dUTP rich-end labeling; FRIM, fluorescent ratio imaging microscopy; SNARF-1, carboxy-SemiNapthoRhoda-fluor-1 acetoxymethyl ester, acetate; FITC, fluorescein isothiocyanate; PBMC, peripheral blood mononuclear cells; PBS, phosphate-buffered saline (10 mM phosphate, 138 mM NaCl, 2.7 mM KCl, pH 7.4);

Reference now will be made in detail to the aspects and embodiments of the invention. Each example is provided by way of explanation of the invention, and not a limitation of the invention. In fact, it will be apparent to those skilled in the art that various modifications, combination, additions, deletions and variations can be made in the present invention without departing from the scope or spirit of the invention. For instance, features illustrated or described as part of one embodiment can be used in another embodiment to yield a still further embodiment. It is intended that the present invention covers such modifications, combinations, additions, deletions and variations as come within the scope of the appended claims and their equivalents.

The methods of the present invention include, but are not limited to, the multiparameter analysis of cells and the separation of cell subpopulations by high-speed cell sorting. Since the measurement of pH₁ can require two fluorescent channels (580 nm and 640 nm) of a flow cytometer, other available channels may be used for additional marker recognition. For example, these other channels, in the case of a flow-cytometer and high-speed cell sorter could be set at 530 nm, typically used for FITC labeling, 670 nm used for APC labeling, and a UV channel, for Hoechst (Ho) 33342 or DAPI staining. Fluorescent compensation software such as the Summit version 2.0 (Cytomation) can allow full use of all of these channels as well as pHᵢ ratiometric measurements in real-time.

Cell subpopulations can be selected based on the presence or absence of cell membrane antigen markers, the intracellular pH, and the cell cycle status. While not bound by any one theory, the higher the pHᵢ of a cell population, the greater the proportion of cells present in S-phase of the cell cycle and vice versa. It is possible to selectively detect the proliferating and non-proliferating populations in real-time and separate them by high-speed cell sorting. Alternatively, it is possible to selectively detect cell populations that are more sensitive to apoptotic-inducing agents, since their pHᵢ would decrease with time with a concomitant increase in the proportion of apoptotic cells that can be determined by, for example, Hoechst Ho33342 staining, annexin-V-FITC or TUNEL labeling. Many other alternative uses are within the scope of the present invention including, but not limited to, simultaneous multiparameter analysis in real-time followed by high-speed cell sorting.

Multiparameter analysis may start with leukemic cell lines and progress to include primary normal and leukemic samples. The methods of the present invention, however, may be applied or adapted to any non-leukemic cell population that might include a subpopulation of proliferating cells. Such cell populations include, for example, cultured cell lines of non-leukemic cells, or cells suspended in a fluid medium, wherein the cells have been isolated from a tissue, the integrity of which has been disrupted to release individual cells or cell clumps.

The cells can be incubated first with pre-determined NHE inhibitors. Thereafter, they can be incubated with annexin-V-FITC, Ho33342 and SNARF. Ho33342 and SNARF, for example, can be added simultaneously and incubated for 30 min at room temperature (RT). Since one of the wavelengths used to measure SNARF is 580 nm, which is also the emission wavelength of propidium iodide, the latter cannot be used to detect necrotic or dead cells. The incorporation of Ho33342, however, allows analysis of the cell cycle characteristics of the population and measurement of the proportion of dead cells. The cell cycle parameters may also be analyzed off-line using appropriate software.

Annexin-V, an FITC-conjugated membrane antigen indicator, may be replaced with other antigen indicators. For example, an antigen indicator conjugated to APC can be used to selectively detect a normal blood stem cell subpopulation. The sensitivity to NHE inhibitors of a cell subpopulation having the selectively detected antigen, can be measured. Aliquots of cells may be labeled with panels comprising more than one biomarker. An example of one such panel incorporates CD38-FITC, CD34-APC, SNARF and Ho33342. Other examples of possible panels can include substituting CD38-FITC with CD117(c-kit)-FITC, with CD91 (Thy-1)-FITC or with AC133-FITC.

The higher the proportion of cells in S-phase of the cell cycle and the higher the pHᵢ, the greater the sensitivity to the inhibitor. Since CD38 defines a mature stem cell population as well as progenitor cells, a CD38⁺CD34⁺ cell population exhibits the highest pHᵢ, the greater the proportion of S-phase cells and, therefore, the highest sensitivity to an NHE inhibitor and highest apoptotic cell number. In contrast, CD91⁺ or AC133⁺CD34⁺ cells are more primitive with a larger number of quiescent cells exhibiting a lower pH₁ and therefore a low proportion of apoptotic cells.

The procedures of the present invention, therefore, can provide techniques to analyze combinations of cell markers as described above, or those specific for other lympho-hematopoietic lineages to differentiate the effects of NHE inhibitors on normal different cell subpopulations.

A similar reasoning can be applied to leukemic cell populations that also show aberrant flow cytometric profiles distinguishable from the normal population. A typical example would be chronic myeloid leukemia in chronic phase. However, in the case of ALL, the leukemic cell population can be defined by a high proportion of CD19⁺ cells. Therefore, CD19 is a biomarker that, when combined with pHᵢ and Ho33342 measurements, can be used to differentiate between leukemic and non-leukemic populations, and between proliferating and non-proliferating populations and in combination with Ho33342 can be used to determine whether proliferating cells are also multiple drug resistant. Using a flow cytometer combined with a cell sorter, these populations can then be separated and analyzed for their sensitivity to the various selected NHE inhibitors. In this particular example, a cell analysis is performed, and the cells sorted, on the basis of a leukemic cell population marker, pHᵢ and cell cycle status. The cells can then be treated with the inhibitor to determine which leukemic cell populations are most sensitive. Alternatively, the cells can be treated with inhibitor prior to analysis to determine which populations have been reduced or eliminated. This strategy is the basis of the purging assay described in Example 18 below. Once sorted, the cells can then be subjected to various NHE inhibitors and the proportion of apoptotic cells determined by using either annexin-V-FITC or pre-labeled Ho33342.

Cell populations sorted on the basis of pHᵢ and cell cycle parameters will also provide starting populations for the in vivo assays described in Examples 19 and 20 below. One aspect of the present invention is to induce apoptosis in selectively identified subpopulations of cells by inhibiting an NHE to kill or inhibit proliferating cells. The cells may be contacted with the NHE inhibitor ex vivo or in vivo. It is contemplated that the ex vivo application of the methods of inducing apoptosis of the present invention may comprise the steps of obtaining a population of cells from a human or animal, identifying therein a subpopulation of proliferating cells according to the methods of the present invention, identifying an effective dose of an NHE inhibitor, contacting the subpopulation of cells with the effective dose of the inhibitor, thereby reducing the pHᵢ of the cells and inducing apoptosis therein, and returning the treated cell population to the human or animal patient. It is further contemplated to be within the scope of the present invention for the identified subpopulation of proliferating cells to be selectively isolated from the cell population and contacted with the NHE inhibitor. The treated cell subpopulation can then be combined with the untreated cells and returned to the patient.

It is still further contemplated that proliferating cells may be treated with an NHE inhibitor in vivo. The in vivo treatment of proliferating cells comprises the steps of obtaining a population of cells from a human or animal, identifying a subpopulation of proliferating cells according to the methods of the present invention, identifying a dose of a pharmaceutically acceptable NHE inhibitor that is effective in inducing apoptosis in the subpopulation of proliferating cells, and administering the effective dose of the pharmaceutically acceptable NHE inhibitor to the human or animal, thereby inducing apoptosis in the subpopulation of cells in vivo in the human or animal patient.

One aspect of the present invention, therefore, is a method of selectively identifying a subpopulation of cells, comprising the steps of obtaining a population of human or animal cells that includes at least one subpopulation of differentially distinguishable cells, contacting the cells with a pH indicator, determining the pHᵢ of the cells, determining the percentage of cells in the S-phase of the cell cycle as a function of the pHᵢ of the cell population, identifying those cells in the population of cells that are in the S-phase of the cell cycle, thereby selectively identifying at least one subpopulation of human or animal cells, and selectively isolating the at least one differentially distinguishable subpopulation of cells.

A suitable pH indicator used in the methods of the present invention for selectively identifying a subpopulation of cells can be, but is not limited to, a fluorescent pH indicator, for example, selected from 1,4-diacetoxy-2,3-dicyanobenzene (ADB), 4-methylumbelliferone (4-MU), 2′,7′-bis-carboxyethyl-5(6)-carboxyfluorescein (BCECF) and carboxy-SemiNapthoRhoda-Fluor-1 acetoxymethyl ester, acetate (carboxy SNARF-1 AM).

In one embodiment of the method of the present invention, the fluorescent pH indicator is carboxy-SemiNapthoRhoda-Fluor-1 acetoxymethyl ester, acetate (carboxy SNARF-1 AM).

Aspects of the method of the present invention can also comprise selectively identifying a population of human or animal cells that may be obtained from a non-cancerous tissue or a cancerous tissue, and can include at least one subpopulation of proliferating cells.

In other embodiments of the method of the present invention for selectively identifying a population of cells, the population of human or animal cells may be selected from a peripheral blood cell population, bone marrow cell population, a leukemic cell line population and a primary leukemic cell line population.

In various embodiments of the method of the present invention for selectively identifying a population of cells, the primary leukemic cell population may be selected from an acute lymphocytic leukemia, an acute myeloid leukemia, a chronic lymphocytic leukemia, a chronic myeloid leukemia and a pre-B acute lymphocytic leukemia.

In still other aspects of the methods of the present invention for selectively identifying a population of cells, the population of human or animal cells can be a leukemic cell line selected from bone marrow acute myelogenous leukemia, acute T-cell leukemia, peripheral blood acute lymphoblastic leukemia, chronic myeloid leukemia, acute monocytic leukemia and B-cell leukemia.

In yet other aspects of the method of the present invention for selectively identifying a subpopulation of cells, the population of human or animal cells may be contacted with at least one cell surface marker indicator capable of selectively binding to at least one differentially distinguishable subpopulation of cells, and selectively isolating the at least one subpopulation of cells binding the at least one indicator.

In one embodiment of the method of the present invention for selectively identifying a subpopulation of cells, the cells are contacted with a cell cycle indicator and a pH indicator, thereby selectively identifying multi-drug resistant proliferating cells. A suitable cell cycle indicator may be, but is not limited to, Hoescht Ho33324.

Another aspect of the present invention is a method for reducing cellular proliferation by inducing cellular apoptosis, comprising the steps of obtaining a population of human or animal cells having a differentially distinguishable subpopulation of proliferating cells therein, contacting the differentially distinguishable subpopulation of proliferating cells with an NHE inhibitor, and inducing a state of apoptosis in the proliferating cells.

In one embodiment of the method of the present invention for reducing cellular proliferation by inducing cellular apoptosis, the method further comprises the steps of identifying the proliferating cells, and selectively isolating the proliferating cells from the non-proliferating cells.

In yet another embodiment of the method of the present invention for reducing cellular proliferation by inducing cellular apoptosis, the method further comprises the steps of contacting the population of human or animal cells with a cell surface antigen indicator capable of selectively binding to a cell surface antigen, identifying at least one cell surface marker of the proliferating cells, and selectively isolating the proliferating cells having the one cell surface marker.

In one aspect of the method of the present invention for reducing cellular proliferation by inducing cellular apoptosis, the population of human or animal cells may be derived from a non-cancerous tissue or a cancerous tissue.

In embodiments of the method of the present invention for reducing cellular proliferation by inducing cellular apoptosis, the population of human or animal cells may be selected from a peripheral blood cell population, bone marrow cell population, a leukemic cell line population and a primary leukemic cell line population.

In other embodiments of the method of the present invention for reducing cellular proliferation by inducing cellular apoptosis, the population of human or animal cells may be a leukemic cell line selected from bone marrow acute myelogenous leukemia, acute T-cell leukemia, peripheral blood acute lymphoblastic leukemia, chronic myeloid leukemia, acute monocytic leukemia and B-cell leukemia.

In still other embodiments of the method of the present invention for reducing cellular proliferation by inducing cellular apoptosis, the population of human or animal cells may be a primary leukemic cell population selected from an acute lymphocytic leukemia, an acute myeloid leukemia, a chronic lymphocytic leukemia, a chronic myeloid leukemia and a pre B acute lymphocytic leukemia.

In embodiments of the method of the present invention for reducing cellular proliferation by inducing cellular apoptosis, the NHE inhibitor may be amiloride or an amiloride derivative. For example, the amiloride derivative may be selected from 5-N,N-hexamethylene)-amiloride (HMA), 5-(N,N-ethyl-N-isopropyl)-amiloride (EIPA), 5-N-methyl-N-isobutyl)-amiloride (MIA), 5-(N-methyl-N-isobutyl)-amiloride (MIBA), simvastatin and phenamil.

In one embodiment of the method of the present invention for reducing cellular proliferation by inducing cellular apoptosis, the NHE inhibitor may be a non-amiloride selected from (2-methyl-5-(methylsulfonyl)-4-pyrrolobenzoyl)-guanidine (EMD), (3-methylsulfonyl-4-piperidinobenzoyl)guanidine methanesulfonate) (Hoe 694), CARIPORIDE™ (Hoe 642), cimetidine, clonidine and hormaline.

In other embodiments of the method of reducing cellular proliferation by inducing cellular apoptosis of the present invention, the method further comprises the steps of contacting the population of human or animal cells with a pH indicator, delivering the human or animal cells to a device capable of detecting the pH indicator, determining the pHᵢ of the human or animal cells from the pH indicator, and determining the percentage of cells of the population of human or animal cells that are in the S-phase of the cell cycle as a function of the pH₁ of the cells.

In embodiments of the method of the present invention for reducing cellular proliferation by inducing cellular apoptosis, the pH indicator may be, but is not limited to, a fluorescent pH indicator, for example selected from 1,4-diacetoxy-2,3-dicyanobenzene (ADB), 4-methylumbelliferone (4-MU), 2′,7′-bis-carboxyethyl-5(6)-carboxyfluorescein (BCECF) and carboxy-SemiNapthoRhoda-Fluor-1 acetoxymethyl ester, acetate (carboxy SNARF-1 AM).

In one embodiment of the method of reducing cellular proliferation by inducing cellular apoptosis of the present invention, the pH indicator is the fluorescent pH indicator carboxy-SemiNapthoRhoda-Fluor-1 acetoxymethyl ester, acetate (carboxy SNARF-1 AM).

In yet another embodiment of the method of reducing cellular proliferation by inducing apoptosis according to the present invention, a dose of an NHE inhibitor effective in inducing apoptosis in a subpopulation of proliferating cells is identified, the subpopulation of cells is contacted with the inhibitor, thereby inducing apoptosis, and the ex vivo treated subpopulations of cells is returned to the human or animal.

In still another embodiment of the method of reducing cellular proliferation by inducing apoptosis according to the present invention, a dose of an NHE inhibitor effective in inducing apoptosis in a subpopulation of proliferating cells is identified, and the effective dose is administered to a human or animal, thereby killing or inhibiting a proliferating subpopulation of cells.

It is also contemplated to be within the scope of the present invention for at least one additional therapeutic agent to be administered to the patient receiving the NHE inhibitor, wherein the at least one additional therapeutic agent may be, for example, an anti-tumor agent, an anti-proliferative agent or any combination thereof that is useful in the treatment of cancer.

Other aspects of the present invention include kits for selectively identifying a subpopulation of cells, comprising packaging material containing a pH indicator, at least one cell surface antigen indicator capable of selectively binding to a cell surface antigen, and instructions for the use of the pH indicator and the at least one cell surface antigen indicator for selectively identifying a subpopulation of human or animal cells.

One embodiment of the kit of the present invention for selectively identifying a subpopulation of cells further comprises a cell cycle indicator and instructions for identifying multi-drug resistant cell subpopulations.

Other embodiments of the kit of the present invention for selectively identifying a subpopulation of cells can further comprise at least one enzyme capable of digesting connective tissue, and instructions for the use thereof for disrupting a tissue.

Yet another aspect of the present invention is a kit for reducing cellular proliferation in a subpopulation of cells by inducing cellular apoptosis, comprising packaging, and containing at least one pH indicator, at least one cell surface antigen indicator capable of selectively binding to a cell surface marker, at least one NHE inhibitor, and instructions for the use of the at least one pH indicator, at least one indicator capable of selectively binding to a cell surface marker, and at least one NHE inhibitor for reducing cellular proliferation by inducing cellular apoptosis.

Another embodiment of the kit for reducing proliferation in a subpopulation of cells by inducing cellular apoptosis further comprises at least one enzyme capable of digesting connective tissue, and instructions for the use thereof for disrupting a tissue.

Yet another embodiment of the kit for reducing cellular proliferation by inducing cellular apoptosis further comprises instructions for the in vivo dose optimization and administration of an NHE inhibitor to a human or animal patient.

The present invention is further illustrated by the following examples, which are provided by way of illustration and should not be construed as limiting. The contents of all references, published patents and patents cited throughout the present application are hereby incorporated by reference in their entirety.

### EXAMPLE 1

### Sources of Cells

(a) Human Samples

Peripheral blood or bone marrow mononuclear cell (PBMC or BMMC) samples were obtained from normal adult donors or from leukemic patients. Samples were obtained from patients with acute lymphocytic and myeloid leukemia (ALL and AML), chronic lymphocytic and myeloid leukemias (CLL and CML) and pre-B-ALL. The mononuclear cell fraction was obtained by Ficoll-Hypaque separation.

(b) Leukemic Cell Lines

The following human leukemic cell lines were obtained from the American Type Culture Collection (ATCC, Rockville, Md.): KG-1a (bone marrow acute myelogenous leukemia), Jurkat (acute T-cell leukemia), Molt-4 (PB acute lymphoblastic leukemia), CEM (peripheral blood acute lymphoblastic leukemia), K562 (chronic myeloid leukemia), THP-1 (acute monocytic leukemia), Nalm-6 (B-cell leukemia). All cell lines were cultured in Dulbecco's Modified Eagles Medium (DMEM) containing 10% heat-inactivated fetal bovine serum (FBS). Only cells in the log phase growth were used.

### EXAMPLE 2

### Growth and Expansion of Primary Leukemic Cells in NOD-SCID Mice

Primary human leukemic cells are typically difficult to grow in culture. The non-obese diabetic, severe combined immunodeficient (NOD/LtSz scid/scid, NOD-SCID, Jackson Laboratories, Bar Habor, Mass.), however, is an established animal model for growing and expanding leukemic cells as subcutaneous nodules, as described by Lapidot et al., Lab. Anim. Sci. 43, 147-150 (1993) and Sinard et al., Blood 87, 1539-1548 (1996), incorporated herein by reference in their entireties. The method of expanding the original leukemic sample containing between 1-5×10⁷ cells to more than 5×10⁸ cells was described in Yan et al., Blood 88, 3137-3146 (1996), incorporated herein by reference in its entirety. Since relatively large numbers of cells were required for flow cytometric analyses (see below), especially those involving both pHᵢ and cell cycle measurements, cells derived from the nodules were used.

The NOD-SCID mice were kept under stringent sterile conditions in a Hepa-filtered mouse rack. Untreated NOD-SCID mice were injected subcutaneously with about 1×10⁷ cells suspended in 100 μl of MATRIGEL™ (Becton Dickinson, San Jose, Calif.) as described by Yan et al. Nodules were usually seen between 4 and 8 weeks after subcutaneous injection and were excised after a maximum of 12 weeks. A cell suspension was prepared after collagenase/dispase digestion of a nodule for 45 min at 37° C., and mechanical disrupting by repeated pipetting.

### Protocol for Producing Leukemic Cells in NOD-SCID Mice

Female NOD-SCID mice were used. Donor and patient cell samples were obtained immediately after peripheral blood and/or bone marrow had been harvested from a patient. The mononuclear cell fraction was prepared by Ficoll separation and the cells were either immediately inoculated into mice or cryopreserved. Samples of the Ficoll-separated fraction were used for cytogenetics, phenotypic analysis and pHᵢ determination. Animals were given up to four 100 μl subcutaneous inoculations (right and left hind and front flanks) each consisting of 1-2×10⁷ cells suspended in cold IMDM mixed with cold MATRIGEL™. After inoculation, exposure to the body temperature of the mouse caused the MATRIGEL™ to gel, thereby immobilizing the cells.

To ascertain whether the cells were growing, the size of the nodule was measured weekly. In mice with multiple nodules, one or more nodules would disappear, but reappear at a later time to grow rapidly into large aggressive nodules, increasing in volume by over 40-fold over 2-3 months, and leading to the dissemination of leukemic cells throughout the animal. Cell counts could reach at least about 5×10⁸ cells/nodule. If nodules had not grown after approximately 8-12 weeks, the animals were sacrificed and the experiment terminated. Mice were sacrificed at the latest after 3 months or when the tumor burden increased above 10% of the body weight. At this time, blood was drawn by heart puncture under ether anesthetic and the mice were killed by cervical dislocation. The nodule(s) were excised and most organs were removed for cytogenetic, phenotypic and immunohistochemical analysis. In particular, bone marrow, spleen and liver were used to assess the presence of normal and leukemic colony-forming cells in methyl cellulose cultures, after preparing single cell suspensions. Nodule cells were freed of MATRIGEL™ and other connective tissue by cutting the nodule into small pieces and incubating the pieces in collegenase/dispase for 45 minutes, followed by repeated pipetting. Each nodule was analyzed individually and only the cells from an individual nodule were pooled.

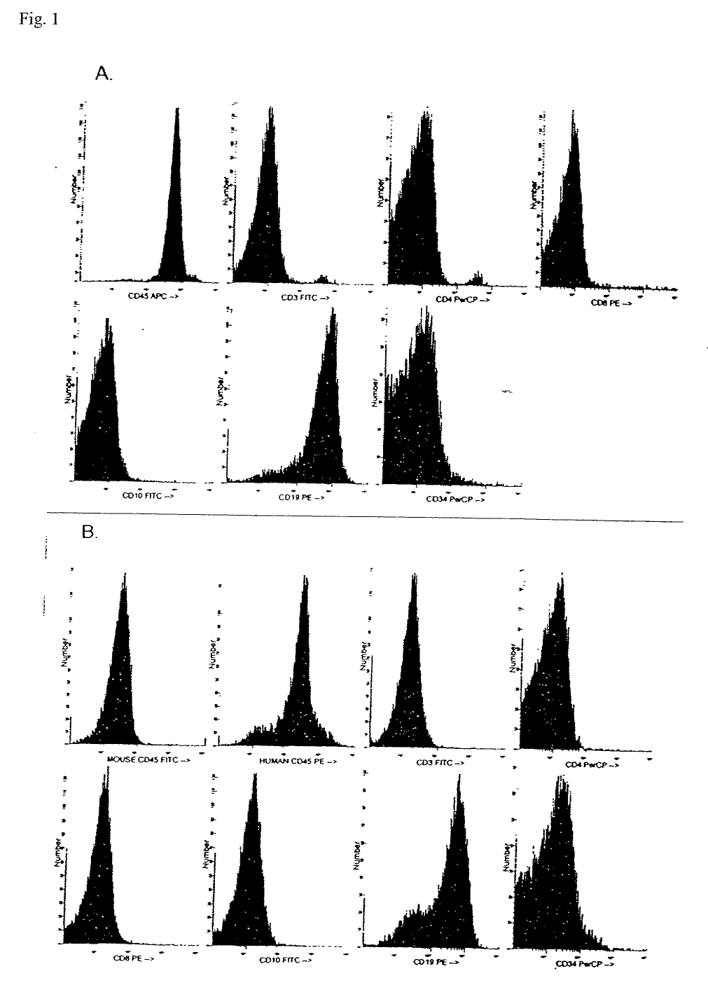

After isolation from a nodule, the cells were analyzed phenotypically and cytogenetically to determine if any changes had occurred from the primary inoculum. Phenotypic characterization was performed using the same panel of monoclonal antibodies employed to define the original sample. In addition, to differentiate and determine the proportion of mouse to human cells present in the nodule, the cells were stained with mouse CD45-fluorescein isothiocyanate (FITC) and human CD45-phycoerythrin (PE) (both obtained from Pharmingen, San Diego, Calif.) and analyzed by flow cytometry. FIGS. 1A and 1B show a phenotypic analysis of the primary inoculum cells obtained from a patient with acute lymphocytic leukemia (ALL) with the phenotype of the cells obtained before (FIG. 1A) and after (FIG. 1B) 3 months of growth in a NOD-SCID mouse. With the exception of the loss of the small populations of CD4 and CD8 cells from the primary inoculum compared to nodule cells, little change occurred in the phenotype of the leukemic cells. In addition, in this particular sample, human CD45⁺ cells comprised more than 85% of the sample (FIG. 1B). Using this method, leukemic cells have been grown and expanded in subcutaneous nodules wherein more than 95% of the cells were human, with massive dissemination of the leukemia into most organs. Cells were not found to have changed either cytogenetically or phenotypically in character. No further purification was performed.

### Treatment of Cells with Phorbol Ester to Induce Na⁺/H⁺ Activity

Normal PBMC were incubated for 5 minutes with 10⁻⁶M phorbol-12-myristate-13-acetate (PMA, Sigma Chemical Company, St. Louis, Mich.) to non-specifically activate the NHE-1. Thereafter the cells were washed with phosphate buffered saline (PBS).

### EXAMPLE 3

### Measurement of pHᵢ by Flow Cytometry and Fluorescence Ratio Imaging Microscopy (FRIM)

For the flow cytometry and FRIM procedures, human peripheral blood or bone marrow mononuclear cells were prepared by Ficoll separation and 1×10⁶ cells and were incubated with 10 μl of a 10 μM carboxy SemiNaphthoRhodaFluor-1 acetoxymethyl ester, acetate (carboxy SNARF-1 AM, Molecular Probes, Eugene, Oreg.) solution for 15 minutes at room temperature. Incubations were performed in PBS in the absence of bicarbonate and serum. Calibration curves were performed using the “nigericin clamp technique” as described in Thomas et al., Biochem. 18, 2210-2218 (1979) and incorporated herein by reference in its entirety. Aliquots of the cell suspension were resuspended in a high K⁺-containing buffer set or “clamped” at a specific pH by the addition of 0.03 μmol/l nigericin, an ionophore that allows exchange of H⁺ for K⁺ ions by abolishing the pH gradient across the cell membrane. When the internal and external K⁺ concentrations are approximately the same, the pH rapidly equilibrates to the pH of the bathing solution.

For flow cytometry, the calibration curve resulted from cell suspensions “clamped” at pH values of 6.6, 6.8, 7.0, 7.2, 7.4 and 7.8; for FRIM, pH values of 6.6, 7.0, 7.4 and 7.8 were used.

Measurement of the pHᵢ by flow cytometry or FRIM utilized the fluorescence ratiometric method described by Chow et al., in: Robinson J. P et al., eds, Curr. Protocols in Cytometry, New York N.Y. John Wiley & Sons, Inc. pp9.3.1-9.3.10 (1997), incorporated herein by reference in its entirety. The pH fluorescent indicator is excited at 488 nm and emits at two different wavelengths, 580 nm and 640 nm. The fluorescence ratio of 640 nm:580 nm was used to estimate the pHᵢ of a cell or cell population. The fluorescence intensity at 580 nm and 640 nm (corresponding to those channels in which phycoerythrin (PE) and peridinin chlorophyll protein (PerCP) were measured) were acquired for 50,000 events. It has a high resolution, discriminating differences of less than 0.1 pHᵢ units. It exhibits a very low background fluorescence. and SNARF-1 shows less than 10% leakage from cells within 2 hours. To measure the pHᵢ of individual hematopoietic stem cell populations, cells were labeled, prior to incubation with SNARF-1, with monoclonal antibodies specific for cell surface antigens and conjugated to fluorescein isothiocyanate (FITC) and allophycocyanin (APC).

### Flow Cytometry

When the pHᵢ was measured as a single parameter by flow cytometry, data was aquired by a FacSort or FACSCalibur (Becton Dickenson, San Jose, Calif.). Data was analyzed using the WinList software program (Verity Software House Inc. Topsham, Me.). The calibration curve was obtained using the TableCurve software program (SPSS Inc., Chicago, Ill.) that also allowed evaluation of the sample pHᵢ values, as described in Rich et al, J. Cell Physiol. 177, 109-122 (1998), incorporated herein by reference in its entirety.

### FRIM

A Zeiss Axiovert 135 microscope equipped for bright field optics and fluorescence imaging analysis using the Attofluor fluorescence analyzer and RatioVision software was used. Cells to be analyzed were labeled as above and transferred to Lab-Tek II chamber slides (Nunc, Naperville, Ill.). The slide was place on a heated microscope stage (37° C.) that reduced variation in pHᵢ measurements. Images and emitted fluorescence were captured by two image intensification charged couple device (ICCD) cameras fitted with filters for 580 nm and 640 nm. Each field of view was divided into 9 regions of interest (ROI) and the fluorescence intensity for each wavelength measured in each of the ROIs simultaneously and in real-time over a period of 10-15 seconds. As many as 3 to 4 different fields per sample were analyzed. The TableCurve software was again used to calculate the calibration curve and evaluate sample pHᵢ values.

### EXAMPLE 4

### Measurement of the pHᵢ of Peripheral Blood Cells and Bone Marrow Mononuclear Cells

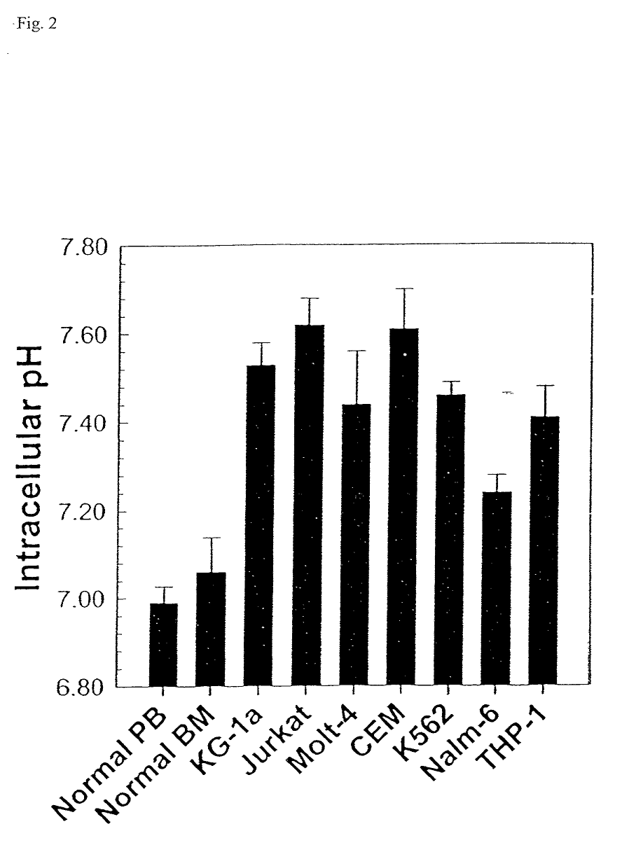

Using flow cytometry and the pH-sensitive fluorescent indicator SNARF, the pHᵢ of normal human peripheral blood (PB) and bone marrow (BM) mononuclear cells was measured. No significant difference (p>0.05) in pHᵢ occurred between PB (pH₁=6.99±0.037, n=25) and BM (pH₁=7.06±0.08, n=10), as shown in FIG. 2. It should be noted that although the pH₁ of specific cell subpopulations could be measured, data is presented in FIG. 2 for whole, i.e. mixed, populations of cells to compare different samples, under the same conditions.

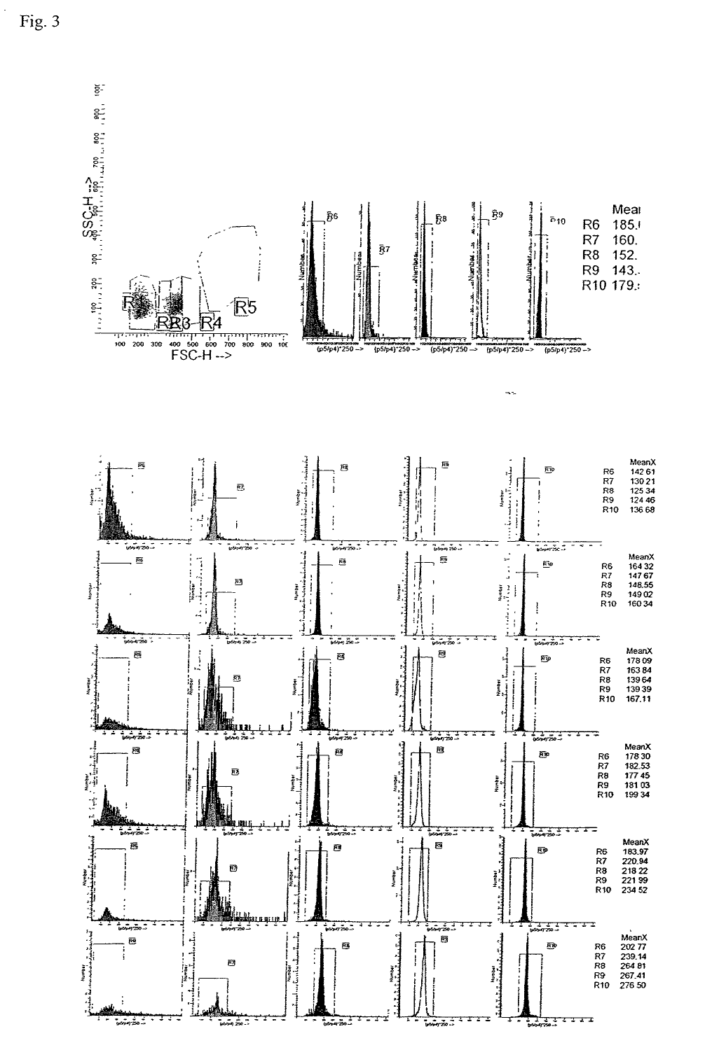

Using donor peripheral blood, the pHᵢ of the whole, ungated cell population was 6.95 (n=17, standard deviation=0.117, standard error=0.037, pHᵢ range= 0.403, pHᵢ minimum=6.78, pHᵢ maximum=7.19). A dot blot of normal peripheral blood is shown in FIG. 3. In this example, the event clusters have been divided into specific populations. The “lymphocyte” population that usually contains all of the CD34⁺ cells was divided into three separate regions (designated R2, 3 and 4). The fluorescent ratio histograms and mean values for each of the regions are shown for regions R6 to R10 representing the dot plot regions R1 to R5 respectively. The pHᵢ values for each region were as follows: R6, 7.46; R7, 7.15; R8, 7.23; R9, 7.11; R10, 7.25. The pH for the standard curve for each region ranges from (top to bottom) 6.8 to 7.8 in 0.2 pH units. The calibration values for each of the regions are also shown in the lower part of the figure. The mean fluorescence ratio increases with increasing pH, leading to a displacement from left to right. The pHᵢ measurements demonstrate that depending on the cell population, different pHᵢ values were obtained.

A more specific extension of this technique is shown in Table 1 wherein the pH₁ of individual hematopoietic stem cell populations was measured.

TABLE 1
pHᵢ of Peripheral Blood Stem Cell Subpopulations

| | CD 9O | | | |
|---|---|---|---|---|
| | (Thy-1) | HLA-DR | CD 38 | CD 34 |
| CD 90 (Thy-1) | 6.79 | | | |
| HLA-DR | | 7.41 | | |
| CD 38 | | | 7.41 | |
| CD 34 | 6.89 | 7.35 | 7.39 | 7.21 |

pHᵢ of normal peripheral blood = 6.95

Cells expressing CD90 and CD34 cell surface antigens are considered the most primitive hematopoietic stem cells, and as such would be expected to be in the Gₒ phase of the cell cycle. In contrast, CD34⁺CD38⁺ and CD34⁺ HLA-DR⁺ cell populations encompass more mature CD34⁺ stem cell populations as well as myelomonocytic and erythropoietic progenitor populations, and would, therefore, be expected to be proliferating populations. The pHᵢ, as a measure of NHE activity, reflected this situation; low NHE activity corresponded to low pHᵢ value, and low proliferative activity; high NHE activity corresponded to a higher pHᵢ, and greater proliferative activity. These data show that the pHᵢ of a specific hematopoietic stem cell population reflected the cell cycle status of that population.

### EXAMPLE 5

### Measurement of the pHᵢ of Leukemic Cell Lines

Various established leukemic cell lines exhibited a higher pH₁ (p<0.01) than did normal PBMC and BMMC (FIG. 2). These observations were similar to those previously reported describing intracellular alkalinization of continuously proliferating cell lines, as in Rebello et al., Exp. Cell Res. 218, 581-585, (1995); Perez-Sala et al, J. Biol. Chem. 270, 6235-6242, (1995), Bischof et al., Biochim. Biophys. Acta 1282, 131-139 (1996), and Larsson et al, Anticancer Res. 9, 1-7 (1989) and incorporated herein by reference in their entireties.

To test whether a greater pHᵢ could be measured in cells undergoing continuous proliferation, compared to resting cells, four different human leukemic cell lines, KG-1a, an acute myeloid leukemic cell line, Nalm6, a B-cell leukemia, K562, an erythroleukemia and CEM, a T-cell leukemic cell line were used. The pHᵢ of each cell line was measured alone or when seeded at different proportions into normal peripheral blood. The pHᵢ of each cell line alone was as follows: KG-1a, 7.30; Nalm6, 7.24; K562, 7.26; CEM, 7.04. The CEM cell line grew at a very slow rate, which appears to be reflected in the pHᵢ measured. Mixing donor peripheral blood with leukemic cell lines so that the proportion of the added cells was 5% or even 1% of the total, resulted in pHᵢ values similar to those obtained with the cell lines alone. Below 1%, the cell lines could not be detected because their dot plot profile could not be distinguished from normal cells. Thus, leukemic cultured cell lines exhibited a greater pHᵢ than normal cells. These results were the groundwork for determining whether primary leukemic cells exhibited a greater pHᵢ than normal cells.

### EXAMPLE 6

### Measuring the pHᵢ of Primary Leukemic Cells

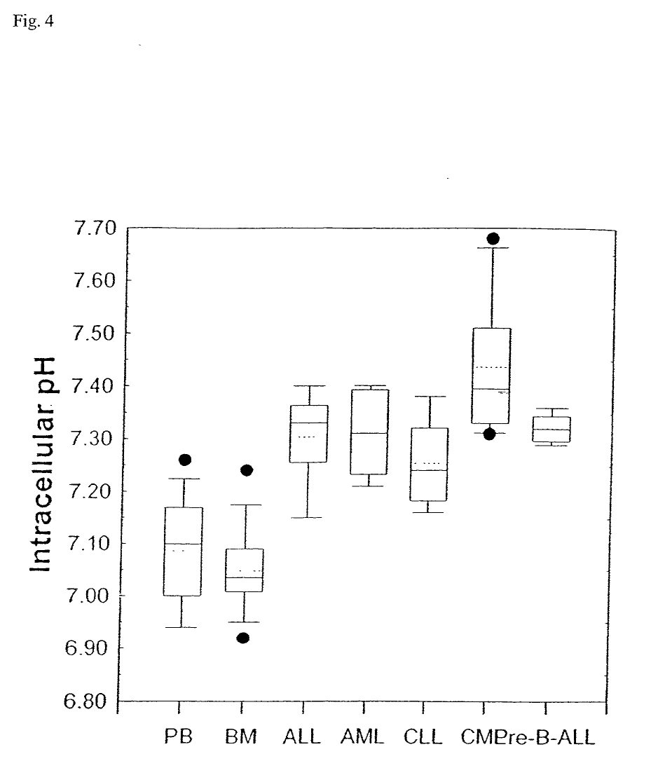

The behavior of primary leukemia samples was examined. FIG. 4 shows the pHᵢ values of samples obtained from patients with different leukemias. In FIG. 4 the lowest and highest boundaries of the box indicate the 25^{th} and 75^{th} percentiles respectively; the whiskers above and below the box designate the 95^{th} and 5^{th} percentiles respectively; the solid line within the box represents the median value while the dotted line is the mean value; dots above or below the box indicate outliers. Acute lymphoblastic leukemia (ALL, n=5), acute myelogenous leukemic (AML, n=5), chronic lymphocytic leukemia (CLL, n=5), chronic myelogenous leukemia (CML, n=6), pre-B-acute lymphoblastic leukemia (pre-B-ALL, n=5). Analysis of variance demonstrated that compared to normal donors, the pH, values from ALL (p= 0.005), AML (p=0.004), CLL (p=0.049), CML (p<0.001), and pre-B-ALL (p= 0.002) patient PBMC were all significantly higher. Primary leukemic cells exhibit a significantly greater pHᵢ than normal cells.

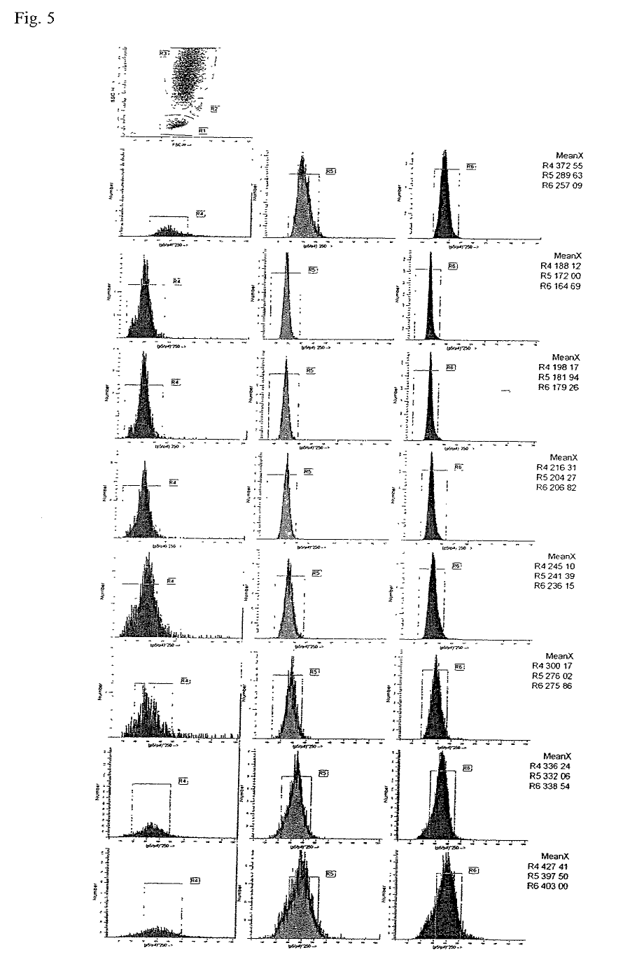

A pHᵢ analysis from a patient with CML in chronic phase with only 2.5% blasts is shown in FIG. 5. The pHᵢ of the regions corresponding to three clusters were determined from the calibration curve. The pHᵢ of 7.68 of R1 of the dot plot shown in FIG. 5 corresponds to R4 of the histogram. The R2 region corresponding to R5 exhibited a pHᵢ of 7.45, while R3 corresponding to R6 showed a pHᵢ of 7.31. In the absence of any regions, the pHᵢ was 7.31. The standards (FIG. 5, top to bottom) range from 6.8 to 7.8 in 0.2 pH units. When the pHᵢ correlated with cell cycle status, the R1 region corresponding to the lymphocyte region, in which all the hematopoietic stem cells were located, was in the active cell cycle.

Another patient with a bifunctional leukemia/lymphoma, exhibited a pHᵢ for the whole ungated population of 7.28, and a pHᵢ of 7.36 for a subpopulation of CD34⁺ cells. In contrast, a patient with an AML with FAB, staging at M0, exhibited a pHᵢ for the ungated population of 7.09, but for a subpopulation of the CD34⁺ cells showing very low forward and side scatter, a pHᵢ of 7.24. These results show that a correlation exists wherein leukemic populations exhibit a higher pHᵢ than normal resting populations. The heterogeneity of leukemia cell types in any one leukemia (as well as in other tumors) requires that each primary cancer cell sample must be analyzed on an individual subpopulation basis.

### EXAMPLE 7

### Measurement of Cell Cycle

The proportion of cells in S-phase of the cell cycle was determined using the propidium iodide technique (CycleTEST Plus DNA reagent kit, Becton-Dickinson, San Jose, Calif.) on 1×10⁶ cells/sample according to the manufacturer's instructions. Data were acquired by flow cytometry, and analyzed using the ModFit LT software program (Verity Software House Inc).

### EXAMPLE 8

### Correlation Between pHᵢ and Cell Cycle Status

The percentage of cells in the S-phase of the cell cycle was determined as a function of the pHᵢ with aliquots of the same cell sample. Prior to the analysis, peripheral blood mononuclear cells (PBMC) were incubated with phorbol ester to activate the NHE-1, as described in Example 2. Samples incubated with phorbol ester for 5 minutes showed a statistically higher (p=0.003) pHᵢ (7.32±0.043) than unstimulated PBMC (7.03±0.04) and showed a statistically greater (p<0.001) proportion of cells in S-phase (8.7%±0.17%) than untreated PBMC (1.19%±0.34%).

The proportion of S-phase cells in populations of normal PBMC or BMMC was less than 5%. Fresh primary AML cells demonstrated some variation in the proportion of S-phase cells, ranging from about 14 to about 20%. Two patients from whom the fresh AML cells were obtained both had blast cell counts of between about 45 and about 50%.

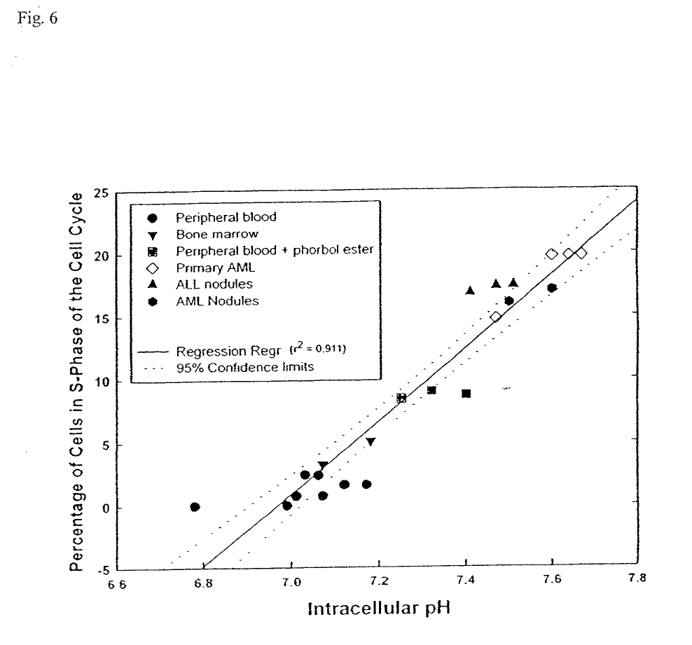

The primary ALL inocula used to obtain subcutaneous nodules contained greater than 75% blasts in the peripheral blood. Both primary AML and ALL cells formed subcutaneous nodules in NOD-SCID animals. When the nodules were excised and the cells analyzed for pH₁ and the proportion of cells in S-phase of the cell cycle, both parameters were increased above normal values. When the pHᵢ and the proportion of S-phase cells were further analyzed for any correlation, a positive linear regression was obtained with a correlation coefficient (r²) of 0.911, indicating that a direct correlation existed between pHᵢ and proportion of cells in S-phase, as shown in FIG. 6. The leukemic cell lines (KG-1a, CEM, K562 and Nalm-6) exhibited both high pHᵢ and a high proportion of cells in S-phase, these parameters being dependent on the growth curve of the cells. Thus, intracellular alkalinization is associated with an increased proliferation. While not bound by any one theory, the unregulated proliferation of leukemic cells is, at least in part, due to suppression of normal apoptotic mechanisms. Accordingly, NHE inhibitors will reduce the pH₁ and induce apoptosis in leukemic cells.

### EXAMPLE 9

### Detection of Cell Subpopulations Characterized by the pHᵢ and Two-Color Membrane Antigen Expression

The first stage in correlating pHᵢ with cell cycle status was to show that the pHᵢ can be detected in specific hematopoietic cell populations derived from a total population of hematopoietic cells. To measure pHᵢ, carboxy-SNARF-1 AM (SNARF-1) was used, as described in Example 3.

By adding antibodies conjugated with FITC or APC directed against expressed membrane antigens, specific cell populations could be singled out and their pHᵢ measured, as shown in Table 1, Example 4. By identifying specific membrane antigens, the “stemness”, or stage of stem cell maturation, of the proliferating leukemic cell population(s) could be defined. This allowed a proliferating leukemic cell population to be defined, not only by a common stem cell antigen, e.g. CD34, but whether it fell into the category of being primitive, e.g. expressing the CD90 (Thy-1) antigen, or more mature, e.g. expressing CD38 or HLA-DR. In addition, the stem cell antigen AC133 defines a subset (0.3%) of CD34⁺ cells. If present on leukemic cells, it may also be used to define a subset population of leukemic cells. Coupled with antibodies that are used to delineate leukemic populations, e.g. HLA-DR, CD19, CD20, CD24,CD10 for different types of ALL and CD13 and CD33 for some of the AML types, this type of analysis provides information with respect to leukemic cell function, and cell cycle status.

### EXAMPLE 10

### Detection of Cell Populations Based on pHᵢ Two Color Membrane Antigen Expression and Cell Cycle

The methodology of Example 8 is also applied to cell cycle analysis so that this factor, as well as the determinations of pHᵢ and membrane antigen expression, can be performed simultaneously. A high-speed cell sorter equipped with an argon, helium-neon and ultra-violet (UV) light laser is used. It has the capacity to measure pHᵢ in real time by the ratiometric technique. This allows the aforementioned analysis to be expanded beyond the analysis of cell cycle, to include cell sorting. This provides the opportunity of sorting and purifying specific cell populations with high pHᵢ and which can be used for later molecular analysis. The inclusion of a UV laser allows measurement of cell cycle parameters using the Hoechst dye 33342. Hoechst 33342, which binds to the AT regions of double stranded DNA, emits a blue fluorescence at 460 nm. Cells that are not in cell cycle are Hoechst ₃₃₃₄₂^{dim}, while those in the proliferative cycle are Hoechst 33342^{bright}.

Hoechst 33342 has also been employed to measure multi-drug resistance, a defect in drug accumulation caused by overexpression of a transmembrane glycoprotein, the P-glycoprotein. If proliferating leukemic cells are not drug-resistant, then they will demonstrate high pHᵢ and be Hoechst 33342^{bright}. Drug-resistant cells, however, even when they are in cell cycle, may still have a high pHᵢ although Hoechst 33342^{dim}. Both of these combinations and results could correlate to the MDR status of the cell.

For this multiparameter analysis, cells are labeled with the monoclonal antibodies specific for cell surface antigens, and stained with a final concentration of 1 μg/ml Hoechst 33342 (Molecular Probes, Eugene, Oreg.) for 1×10⁶ cells for 20 mins at 37° C. in the dark. After briefly washing the cells in medium (IMDM), the cells are loaded with SNARF-1, and data acquisition performed as soon as possible. Coupled with antibodies that delineate a leukemic population, as described in Example 6 above, the simultaneous measurement of pHᵢ and cell cycle parameters provide a tool by which individual subpopulations of cells can be analyzed and sorted, yielding subpopulations of proliferating leukemic cells. This multiparameter cell sorting provides a second alternative to producing proliferating leukemic cell populations using the NOD-SCID mouse. The multiparameter cell sorting capability of the machine is about 20,000 events/sec with 95% purity.

### EXAMPLE 11

### Measurement of Apoptosis by Annexin-V and TUNEL

Two methods were used to determine if a reduction in pHᵢ induced apoptosis. The first was a flow cytometric assay using annexin-V conjugated to fluorescein isothiocyanate (FITC) (Pharmingen, San Diego, Calif.) as a marker for the translocation of phosphatidylserine (PPS), normally located within the plasma membrane. At the onset of apoptosis, PPS is translocated to the outside of the cell membrane, and is available to bind to annexin V in the presence of Ca²⁺ ions. The PPS remains on the surface even when the cells have died, so that a cell in the process of apoptosis cannot be distinguished from one that is already dead. The presence of PPS, as indicated by annexin V binding, can identify early-stage apoptotic cells. Cells were counter-stained with propidium iodide (PI) to determine the proportion of necrotic cells. Cell staining was performed according to the manufacturer's instructions. Nevertheless, by using annexin-V conjugated to fluorescein isothiocyanate, together with SNARF-1 to measure pHᵢ and Hoechst 33342 to measure cell cycle status, a powerful and simultaneous analysis of all three parameters was possible.

A second technique called the TUNEL method was used after termination of cell cultures and, where required, substantiation of apoptosis by the annexin V technique. This could be used as a flow cytometric assay or as an in situ detection of apoptosis. Since apoptotic cells contained fragmented DNA, this could be labeled and detected. TUNEL denotes Terminal deoxynucleotidyl transferase (TdT)-mediated dUTP- Nick End-Labeling (Boehringer Mannhein, Germany) and was performed according to the manufacturer's instructions. Data acquisition was performed by flow cytometry and analyzed using WinList software. The dUTP was conjugated to a fluorochrome or digoxigenin, depending on the kit used. Cells from the cultures (with the exception of those from methyl cellulose) were cytospun (2×10⁵ cells) onto glass slides, fixed in 4% methanol and permeabilized with 0.2% Triton X-100 in PBS for 5 minutes. After washing and equilibration in buffer, the cells are incubated with FITC 12-dUTP and TdT enzyme for 1 hour at 37° C. The cells were then washed and stained with propidium iodide. Using fluorescence microscopy, apoptotic cells appear green, while live cells that had taken up the propidium iodide were red.

### EXAMPLE 12

### Treatment of Cells with Ammonium Chloride or 5-(N,N-hexamethylene)-amiloride (HMA)

To induce intracellular acidosis, cells were incubated in 20 mM ammonium chloride (NH₄Cl) solution for varying periods of time. Thereafter, the cells were washed twice in PBS, labeled with SNARF and the pH₁ measured by FRIM. To inhibit the NHE-1, the amiloride analogue, HMA (Sigma Chemicals, St. Louis, Miss.) was used. A stock solution of 10⁻²M was prepared by dissolving the solid in dimethylsulphoxide (DMSO). Further dilutions were prepared in Dulbecco's Modified Eagles Medium (DMEM). Cells were incubated at concentrations ranging from 10⁻⁶M to 10⁻³M for varying periods of time specified in the results. After incubation, cells were washed and aliquoted according to the type of measurement (pH₁ or apoptosis) performed. Controls included cells not treated with HMA. Previous studies demonstrated that the solvent vehicle (DMSO) at the concentrations of HMA added had no adverse effect on pHᵢ measurements or apoptosis.

### EXAMPLE 13

### RNA Degradation as a Means of Determining the Apoptotic-Inducing Effect of NHE Inhibitors

Total RNA was isolated from KG-1a leukemic cells for varying time intervals (30 min, 1, 2, 3 and 5 h). Total RNA was isolated from drug-treated cells using TRIZOL™ reagent, essentially according to manufacturer's instructions. Briefly, pelleted cells were lysed by the addition of Trizol reagent, and thoroughly homogenized. After the addition of chloroform, the organic and aqueous phases were separated by centrifugation, and RNA contained in the aqueous phases was precipitated with isopropanol. RNA was harvested by centrifugation, washed in 75% ethanol, reprecipitated, and air-dried. Pelleted RNA was redissolved in water and analyzed by spectrophotometry prior to gel analysis. RNA samples (2 μg) were resolved by submarine electrophoresis on ethidium bromide-stained with 1.2% denaturing agarose gels. Gels were visualized using a FMBIO II scanning laser fluorescent imaging system. Under denaturing conditions, the 18S and 28S ribosomal RNA species in intact samples appeared as discrete, intensely stained bands that acted as indicators of overall RNA quality. Drug-induced RNA degradation was indicated by a decrease in the intensity and resolution of the 18S and 28S bands, accompanied by the appearance of a characteristic smear of low molecular weight RNA.

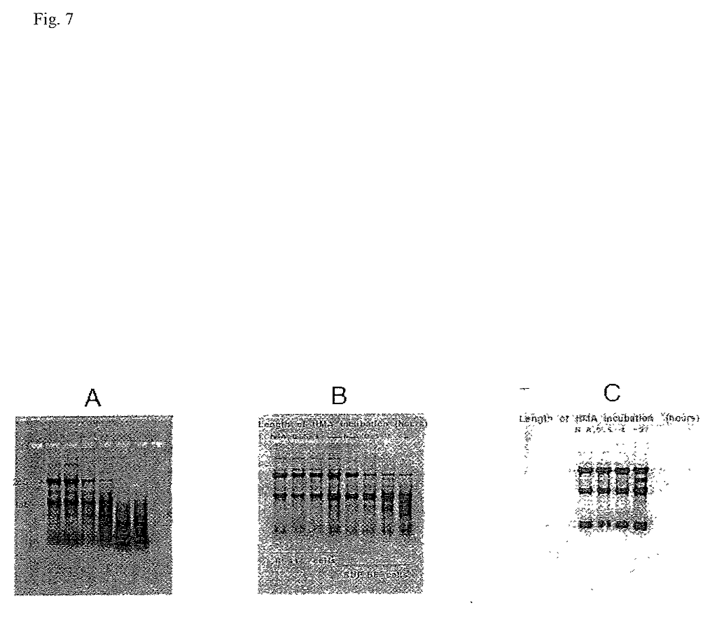

The effects of HMA upon RNA integrity with these different leukemic cell lines are shown in FIGS. 7A-C. Gel analysis of total RNA samples isolated from HMA-treated KG-1a cells indicated that there was significant degradation of total RNA within only one hour of HMA addition, as judged by the intensity of the 18S and 28S ribosomal RNAs. These species of RNA are accepted barometers of overall RNA integrity. By two hours, RNA degradation was almost complete. SUP-B8 and MOLT-4 leukemic cells also affected during the first hour by in vitro HMA treatment. Degradation of 28S and 18S RNA was completed by 3 hours. No significant RNA degradation was seen within the first 2 hours with RS411 leukemic cells, implying that these tumor cells are resistant to the apoptotic-inducing effects of NHE inhibitors.

These observations illustrate the onset of apoptosis upon exposure of the cells to HMA and defined a time window within which apoptosis-inducing genes were up-regulated.

### EXAMPLE 14

### The Effect of NHE Inhibition on the pHᵢ and Apoptosis of Normal Peripheral Blood Mononuclear Cells

Normal peripheral blood cells were incubated in the absence or presence of the NHE inhibitor 5-(N,N-hexamethylene)-amiloride (HMA), at concentrations ranging from 10⁻⁶M to 10⁻³M, for up to 5 hours. Cells were then aliquoted so that pHᵢ and apoptotic measurements could be performed. In these experiments, the pHᵢ was measured using both flow cytometry and fluorescence ratio imaging microscopy (FRIM).

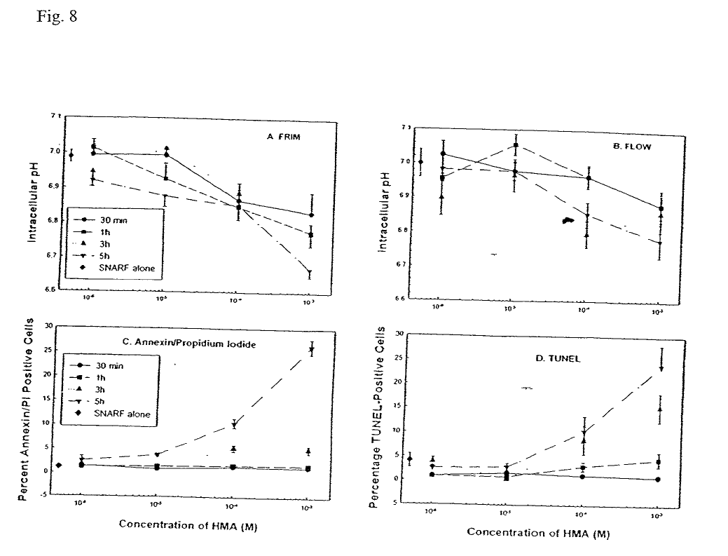

FIGS. 8A-D show that when using both FRIM and flow cytometry, there is a very gradual increase in the pHᵢ with the induction of peripheral blood mononuclear cells (PBMC) for a period of 5 hours, in the absence of HMA. FIGS. 8A and 8B show that the greatest inhibition occurred at 5 hours, using the highest HMA concentration (10⁻³M). Likewise, the greatest proportion of apoptotic cells, as measured by annexin-V and TUNEL labeling, was found after 5 hours incubation with 10⁻³M HMA, as shown in FIGS. 8C and 8D. The proportion of necrotic cells measured by propidium iodide (PI) remained constant at about 5% in the absence or presence of HMA for each time point measured.

Flow cytometric profiles showed that the proportion of annexin-V/PI-positive cells (FIG. 8C) was due to increasing numbers of cells first becoming annexin-V positive and then double-positive for both dyes. All cells underwent apoptosis before dying and were not necrotic beforehand. Using both annexin-V and TUNEL methods, a maximum of approximately 25% of normal peripheral blood cells were apoptotic when the pH₁ was reduced to about 6.8.

### EXAMPLE 15

### The Effect of NHE Inhibition on the pHᵢ and Apoptosis of Leukemic Cell Lines

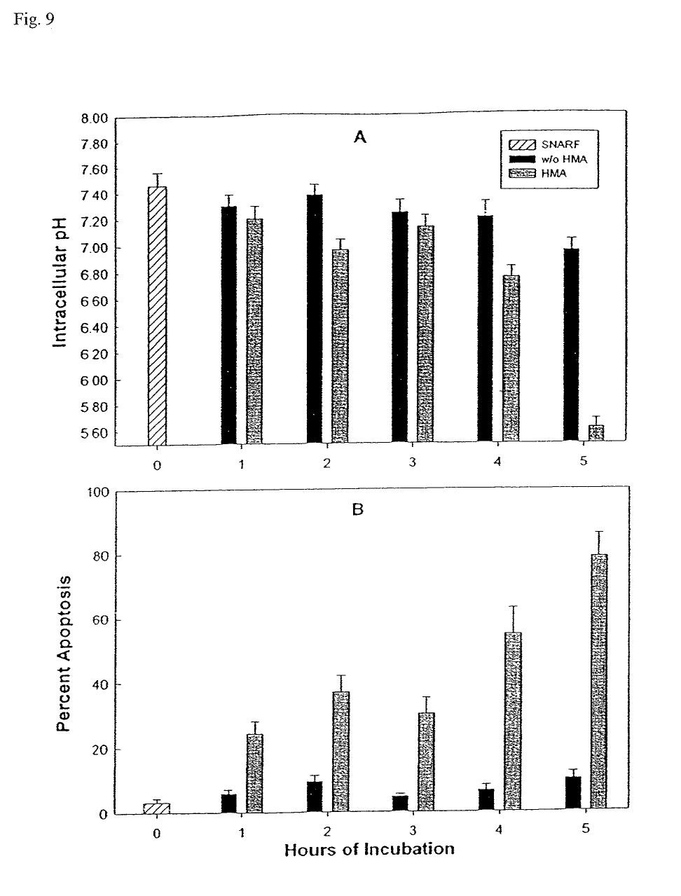

Established leukemic cell lines (KG-1a and CEM) were incubated for varying periods of time with 10⁻³M HMA and pHᵢ and apoptosis measured. FIGS. 9A and 9B show the results for KG-1a cells. For both cell lines, in the absence of HMA, the pHᵢ ranged from 7.43±0.1 at time zero to 6.9±0.08 after 5 hours of incubation. However, after 5 hours with HMA, the pHᵢ had decreased to 5.6±0.07 (n=4) for KG-1a and 6.1±0.06 (n=4) for CEM cells, as shown in FIG. 9A. In the absence of HMA, the percentage of KG-1a and CEM cells undergoing apoptosis, measured by annexin-V, ranged from 2% to 9% for the duration of the experiment, whereas by 5 hours in the presence of HMA, the percentage of KG-1a or CEM cells undergoing apoptosis ranged from about 70% to about 80%, as shown in FIG. 9B.

To demonstrate that reduction in pHᵢ was due to specific inhibition of the NHE by HMA, KG-1a cells were subjected to intracellular acidosis by treatment with 20 mM NH₄Cl for up to 5 h. The results demonstrate that in contrast to cells incubated in the absence of NH₄Cl, there is a gradual, but expected decrease in pH₁ with time. These results demonstrate that HMA is not affecting a pathway other than the inhibition of the NHE. They also indicate that apoptosis can be initiated below a critical pH₁.

### EXAMPLE 16

### The Effect of NHE Inhibition on pHᵢ and Apoptosis of Primary Leukemic Cells

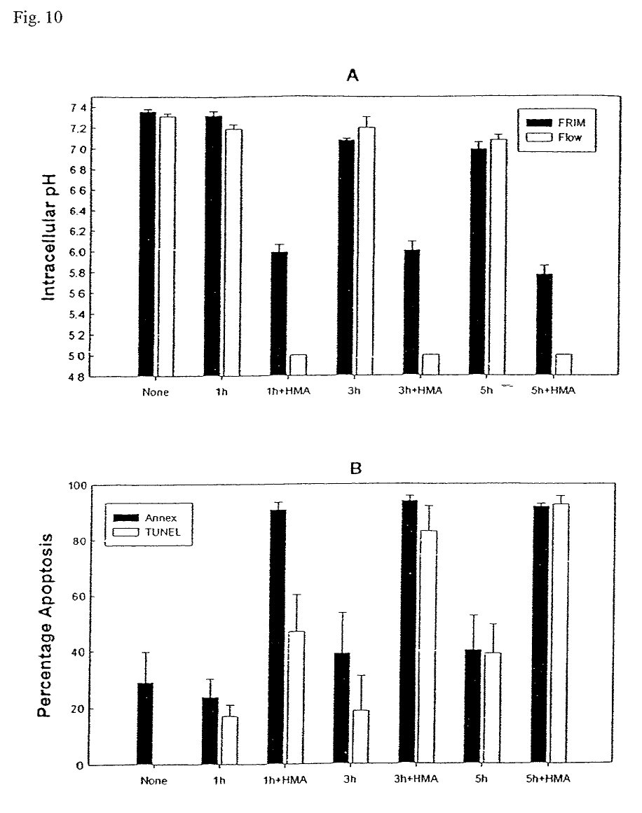

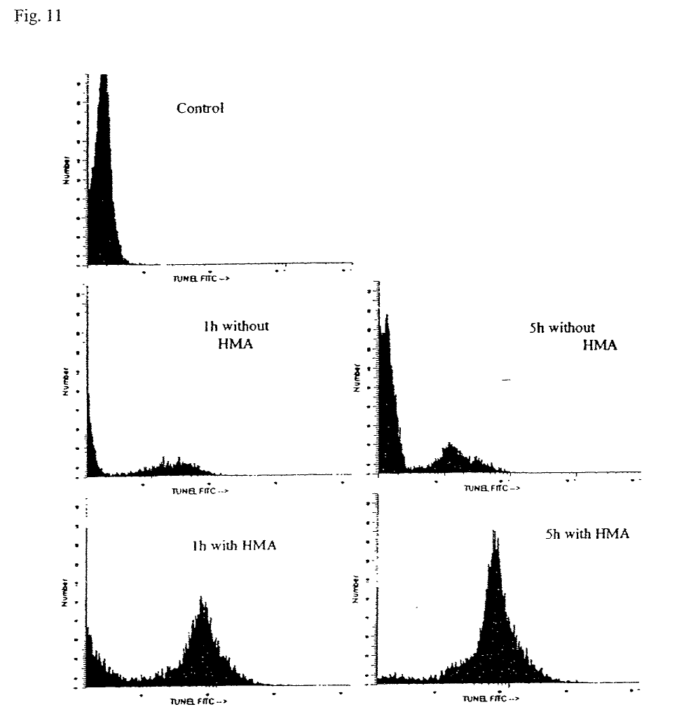

The effect on the pH₁ and apoptosis after incubating fresh ALL patient leukemic PBMC with 10⁻³M HMA for up to 5 hours is shown in FIGS. 10A and 10B. All leukemic samples used in these experiments contained between 85% and 95% blast cells. Intracellular pH was measured by both FRIM and flow cytometry (FIG. 10A), while apoptosis was measured by annexin-V and TUNEL (FIG. 10B). In the presence of pharmacological doses of HMA, pHᵢ was rapidly reduced compared with cells incubated in the absence of HMA. In fact, in all cases, pHᵢ of the cells measured by flow cytometry was too low to measure on the calibration curve. For this reason, pH₁ values for 1 hour, 3 hours and 5 hours were set at 5.0. After 1 hour, the proportion of cells incubated with HMA undergoing apoptosis was about 85% using annexin-V and about 60% using the TUNEL technique. After 5 hours incubation, both methods indicated that over 90% of the cells were apoptotic, as shown in FIG. 10A. FIG. 11 illustrates the flow cytometric histogram profiles for a representative sample of ALL cells incubated in the absence or presence of HMA followed by analysis of apoptosis using the TUNEL method. Thus, primary ALL cells showed greater sensitivity to HMA than normal PBMC, thereby demonstrating an important differential effect between normal and acute lymphocytic leukemic cells.

### EXAMPLE 17

### In Vitro Assays to Determine the Differential Sensitivity and Growth Potential of Normal and Leukemic Cells Treated with NHE Inhibitors

Primary leukemic cells are difficult to grow and maintain in suspension culture. However, leukemic cells can be grown as colonies in a semi-solid medium such as agar or methyl cellulose. The culture system involved mixing together serum (fetal bovine serum, FBS, horse serum, HS, or human AB serum) or serum-free components such as bovine serum albumin, (BSA) with transferrin, β-mercaptoethanol or α-thioglycerol (to reduce oxygen toxicity), growth factors, cells, medium and water-soluble methyl cellulose. The mixed components were then transferred to Petri dishes and incubated for various periods of time, depending on the type of stem or progenitor colony to be assayed. The colonies were counted under an inverted or dissection microscope and results given as a concentration, e.g. number of colonies/10⁵ cells plated. Variations to the normal colony-forming assays provide greater plating efficiency. 35 mm Petri dishes that have 4 small round wells pressed into the plastic were used. Each of the four wells was filled with 100 μl of mixed culture components, so that each assay is performed in quadruplicate. The total culture volume required to fill all 4 wells was 600 μl, compared to at least 4.5 ml if four normal Petri dishes containing 1 ml each were used. To increase plating efficiency, cultures are incubated under low oxygen tension. The culture of hematopoietic cells under 3.5 to 7.5% oxygen, increased the plating efficiency by decreasing the oxygen toxicity in the cultures.

In one set of experiments colony-forming assays of normal and leukemic cells are grown in the absence or presence of different concentrations of pre-defined NHE inhibitors. In a second set of experiments normal and leukemic cells are pre-incubated with NHE inhibitors at different concentrations and for varying periods of time, followed by colony-forming assays. Thus, whereas in the first type of experiment, NHE inhibitor sensitivity is determined, in the second, the growth potential of remaining cells is established.

Normal and leukemic mononuclear cells at a final concentration of between about 1×10⁴ and about 1×10⁵ cells/ml are mixed with METHOCULT™ (Stem Cell Technologies, Vancouver, Canada) in the presence of NHE inhibitor at final concentrations ranging from about 1×10⁻⁸M to about 1×10⁻³M, obtained by serial dilution from a 10⁻²M stock solution. Depending on the vehicle in which the inhibitor is dissolved, the corresponding dilution of the vehicle is added to control cultures. Other controls required for this type of experiment are (a) no addition of either vehicle or inhibitor and (b) no addition of growth factors. The latter controls for any spontaneous colony formation that may occur.

Normally METHOCULT™ is obtained with growth factors included, but the separate components can also be obtained individually. This has relevance to the type of leukemic cell that is being cultured. For example, if a T-cell or B-cell leukemia is cultured, then IL-2 or IL-4 may be required respectively. For a myeloid leukemia, GM-CSF, SCF and IL-3 may be required. When a combination of growth factors is simultaneously added, different colony types may be produced at different times during the life of the culture. Therefore differential colony assessment are performed at 7 days, 10 days and 14 days after the cultures have been initially seeded. Although replicate experiments are performed, unlike murine colony-forming assays, assays involving human cells are notoriously difficult to standardize due to wide donor variation. Therefore, it is necessary to obtain a historical control of normal values (> 10 normal donors) in order to produce a normal range of colony values that can be compared with leukemic values. The production, or rather lack of production, of colony formation produced by normal or leukemic cells in the presence of NHE inhibitors provides an indication of the differential sensitivity of these cell types to these agents.

The second type of assay mentioned above involves pre-incubating normal and leukemic cell samples with NHE inhibitor concentrations for pre-determined time periods. Concentrations of NHE inhibitor ranging from about 10⁻⁶M to about 10⁻³M, at times ranging from 0 hours to about 5 hours, may be used in one instance. In another, the concentrations remain constant, but the time period changes. After pre-incubation, the cells are washed in PBS and the remaining growth potential assayed by colony-forming assays.

In addition to these experiments, in which total mononuclear cells are used, similar experiments on both the above themes are performed using cell subpopulations analyzed and separated by flow cytometry and high-speed cell sorting. Since fewer cells are available, but with an increased level of purity, lower plating cell concentrations can be used.

### EXAMPLE 18

### Purging Assays to Remove Leukemic Blast Cells

The purging assay is a variation of the second set of experiments described in Example 17 above. In this case, however, the method primarily requires flow cytometry. One of the routine clinical assays performed on samples from patients is a cell analysis by flow cytometry that determines the phenotype of the disease. Normally, adequate cell numbers are available for further characterization. Once a phenotype has been determined, the cells are incubated with NHE inhibitors. After a pre-determined time period, the cells are re-phenotyped. In this way, an indication of the proportion of leukemic cells remaining after this purging process is ascertained and therefore the efficiency of cell killing by the NHE inhibitors.

The assay can also be performed in an alternative manner. For example, when the sample has been obtained from a CML, Philadelphia chromosome positive patient (Ph⁺) containing the BCR/ABL translocation, an RT-PCR can be performed before and after NHE inhibitor treatment to assess the proportion of remaining Ph⁺ cells. This provides a far greater sensitivity than does the phenotypic analysis, but it is also more time-consuming.

### EXAMPLE 19

### In Vivo Assays to Assess the Effects of NHE Inhibitors on Normal Hematopoiesis

The assays required to assess the apoptotic effect and efficiency of cell killing by NHE inhibitors also include in vivo assays. In the above Examples, all the information acquired has been on cells manipulated ex vivo. For NHE inhibitors to be administered to a human or animal to inhibit the proliferation of leukemic cell, their effects must be assessed using in vivo models. It is also necessary to assess these effects under both normal and leukemic conditions.

To assess the effects of NHE inhibitors on leukemic cells in vivo, the NOD-SCID model is used. This model provides valuable information on the short- and long-term in vivo effects of NHE inhibitors on normal hematopoiesis. For the experiments, normal C57BL/6J female mice, 6-8 weeks old, are used to determine the dosage, route of administration and time course of NHE inhibitors on steady-state hematopoiesis.

Initial experiments lay the groundwork for the dosage, route of administration and time of the drug effects. For the amiloride NHE inhibitor, the oral LD₅₀ in mice is 56 mg/kg. In humans, the half life of amiloride hydrochloride is 6-9 hours in patients with normal renal function. The normal dose in humans is 5-20 mg/day while in children (6-20 kg) a dose of 0.625 mg/kg has been used. Groups of 3 mice are treated initially with either physiological saline or a single dose of 1 μg, 10 μg or 100 μg and 1 mg/kg body weight of the NHE inhibitor. As additional controls, mice are also injected with a corresponding concentration of the vehicle in which the agent was dissolved. In these first experiments, animals are sacrificed after 1, 2, 3, 5, 7, 10 and 14 days and 3, 4, 6 and 9 weeks. Data are obtained from individual mice and not pooled. At each time point peripheral blood is drawn and the femora, tibia and spleen are removed. Although emphasis is placed on the effect on hematopoiesis, the kidney, gut, liver and lung are removed and prepared for microscopic and immunohistochemical examination, primarily to detect apoptosis. An aliquot of the hematopoietic cell suspensions is used to measure pH₁ and apoptosis, the latter by the annexin-V and TUNEL assays as described in Example 11 above. Another aliquot of the cells is assayed for stem and progenitor cells using in vitro colony-forming assays to assess the effect of the drugs on normal hematopoiesis. A third aliquot is used for morphological and immunohistochemical examination. Finally, RNA is prepared and used for cDNA array profiling in order to determine the pharmacogenomic effect of the drugs on various intracellular systems. The results are compared with animals that have not received drug administration. These experiments provide information to determine whether the differential sensitivity to inhibitors observed in vitro for normal and leukemic cells is also seen in vivo. The maximum dose tolerated by the C57BL/6J mice without significant effects on normal hematopoiesis is used as the minimum starting dose in leukemia-burdened NOD-SCID mice.

In a further series of experiments, the dose and route of administration found to be optimal for a single injection is used to assess the influence of repeated doses of the NHE inhibitor, initially up to 6 days. All experiments have the same end points, namely to determine the kinetics of hematopoietic stem, progenitor and precursor cell populations after NHE inhibitor insult. The results from these experiments impact on the overall assessment of whether a differential effect to NHE inhibitors exists between normal and leukemic cells.

### EXAMPLE 20

### In Vivo Assay to Assess the Anti-Leukemic Effect of NHE Inhibitors Using the NOD-SCID Mouse Model

The non-obese diabetic, severe combined-immunodeficient (NOD-SCID) mouse is used to detect normal primitive human hematopoietic stem cells, as well as to grow leukemic cells. See, for example Larochelle et al, Nature Medicine 2, 1329-1337 (1996); Lapidot et al., Lab. Anim. Sci. 43, 147-150 (1993); Sirad et al., Blood 87, 1539-1548 (1996), incorporated herein by reference in their entireties. The present model is based on techniques developed by Yan et al., Blood 88, 3137-3146 (1996) and McGuirk et al., Bone Marrow Transplant 22, 367-374 (1998), both incorporated herein by reference in their entireties, in which leukemic cells can be grown and expanded as subcutaneous nodules in non-obese diabetic severe combined immunodeficient (NOD-SCID) or SCID mice.

The following technique was used, in part, to obtain results showing that a direct correlation exists between the pHᵢ and the cell cycle status of a population. The technique uses the mononuclear cell (MNC) fraction from patient peripheral blood or bone marrow cells, prepared by Ficoll separation. Approximately 1-2×10⁷ cells were suspended in 100 μl of Matrigel (Becton-Dickinson) diluted 1:3 with IMDM and injected subcutaneously into the flanks of untreated NOD-LtSz scid/scid (NOD-SCID, Jackson Laboratories, Bar Harbor, Me.) mice. Multiple subcutaneous inoculations were given to a single animal, in which case multiple nodules could be obtained. Typically, both left and right hind flanks were injected with sample cells. Prior to injection, an aliquot of the MNC fraction was used for cytogenetics, phenotypic analyses, pHᵢ and apoptosis determinations and cell cycle analysis.

Growth of primary leukemic cell nodules usually occurred after 4 to 8 weeks. Animals were observed at least twice a week for nodule growth, as determined with calipers. In the absence of any prior treatment, an engraftment rate, which is dependent on the type of leukemia inoculated, of between about 25% and about 30% was obtained. After pre-treatment with a sublethal, 300 rad irradiation dose, the engraftment was found to increase to between about 40% and about 45%. In general, aggressive leukemias with high blast numbers exhibited more than 80% engraftment, while chronic phase CML demonstrated an engraftment rate of less than 20%. A maximum tumor burden of 10% of body weight was allowed. For this reason, mice were sacrificed no later than 3 months after injection of leukemic cells.

Peripheral blood was drawn by heart puncture under ether anesthetic. After carbon dioxide asphyxiation, nodules were excised, and the femora and tibia, spleen, liver, gut, kidney and lung removed. Nodules, which were well vascularized, were cut into small pieces and the connective tissue digested with a collagenase/dispase solution for 45 min. at 37° C. Cell counts greater than about 5×10⁸ cells/nodule have been obtained. Cells were then subjected to morphologic, cytogenetic, phenotypic, and immunohistochemical analyses. In addition, pHᵢ, apoptosis and cell cycle analysis was performed on the cells.

Aliquots of nodule cells were also re-inoculated into secondary NOD-SCID hosts, which usually engrafted rapidly and produced secondary nodules within three to four weeks. FIG. 1 depicts a flow cytometric analysis of peripheral blood from an ALL patient prior to injection and after retrieval of the cells from a subcutaneous nodule. In both cases, a panel of monoclonal antibodies were used to determine the phenotype of the leukemia. The data was shown for several cell populations present in the initial inoculum and after retrieval from the nodule. Although there was loss of CD3⁺ and CD4⁺ cells after 3 months growth in a NOD-SCID mouse, there was little phenotypic change in the cells. The CD19⁺ population, typical of ALL leukemia is seen prior to and after injection. In addition, to distinguish between the mouse and human cells present in the nodule they were also stained for the mouse and human CD45 antigens. The lower panel of the figure shows that the nodule contained mostly human CD45⁺ cells. In this example, more than 80% of the cells present in the nodule were human CD45⁺. These, together with cytogenetic results, indicated that little change occurred in the cell population during growth and expansion in NOD-SCID mice and provide a basis for using this model to test NHE inhibitors as anti-leukemic agents.

Approximately 10⁶ primary patient leukemic cells are mixed with Matrigel in a total volume of 100 μl and injected into the flanks of a sublethally irradiated NOD-SCID mice. Depending on the number of cells obtained from the patient, 4-6 animals per patient sample are injected, the minimum number being two. Pairs of animals are followed, one of which receives the drug while the other acts as a control without drug administration. Animals are monitored for the growth of a palpable subcutaneous nodule. Once detected, animals are treated with NHE inhibitors and observed over time. Starting with an initial single drug dose, when no effect is observed within a specific period of time, e.g. one to two weeks, a dose escalation or repeated regimen is assessed using the same route of administration. The end point is abrogation or a reduction in size of the nodule, compared to the paired control.

To monitor the effects of compounds, biopsies of the nodule contents are taken by syringe aspiration and analyzed for the induction of apoptosis. Since only small volumes are obtained, these samples are analyzed primarily by microscopic evaluation using annexin-V staining. All experiments are terminated after 3 months, or if the nodules appear to fail to respond to the NHE inhibitor. Dose regimens differing substantially from those found to have no effect on normal mice are reevaluated for adverse effects on normal hematopoiesis in C57BL/6J mice. The nodules are excised and the organs and tissues analyzed. For each pair of animals, the pHᵢ, cell cycle and degree of apoptosis are measured for the nodule, spleen and bone marrow cells. Aliquots are used for chimerism, phenotypic and cytogenetic analysis. Morphological and immunohistochemical evaluation are performed on cell suspensions from the nodule and hematopoietic organs and on tissue sections of all organs.

What is claimed is:

1. A method of selectively identifying a subpopulation of cells, comprising the steps of:
(a) obtaining a population of human or animal cells having at least one subpopulation of differentially distinguishable cells therein;
(b) contacting the population of human or animal cells with a pH indicator;
(c) determining the pH₁ of the population of human or animal cells;
(d) determining the percentage of cells of the population of human or animal cells in the S-phase of the cell cycle as a function of the pHᵢ of the human or animal cell population; and
(e) identifying the human or animal cells in the population of cells that are in the S-phase of the cell cycle, thereby differentially distinguishing at least one subpopulation of human or animal cells.

2. The method of claim 1, further comprising the step of selectively isolating the at least one subpopulation of differentially distinguishable cells from the population of human or animal cells.

3. The method of claim 1, wherein the pH indicator is a fluorescent pH indicator.

4. The method of claim 3, wherein the fluorescent pH indicator selected from 1,4-diacetoxy-2,3-dicyanobenzene (ADB), 4-methylumbelliferone (4-MU), 2′,7′-bis-carboxyethyl-5(6)-carboxyfluorescein (BCECF) and carboxy-SemiNapthoRhoda-Fluor-1 acetoxymethyl ester, acetate (carboxy SNARF-1 AM).

5. The method of claim 3, wherein the fluorescent pH indicator is carboxy-SemiNapthoRhoda-Fluor-1 acetoxymethyl ester, acetate (carboxy SNARF-1 AM).

6. The method claim 1, wherein the population of human or animal cells comprises at least one subpopulation of proliferating cells.

7. The method of claim 6, wherein the proliferating cells have a pHᵢ between about 0.1 and about 0.6 pH units greater than the pHᵢ of non-proliferating cells.

8. The method of claim 1, wherein the population of human or animal cells is derived from a tissue, and wherein the tissue is disrupted to yield a suspension of cells.

9. The method of claim 1, wherein the population of human or animal cells is selected from a peripheral blood cell population, bone marrow cell population, a leukemic cell line population and a primary leukemic cell line population.

10. The method of claim 9, wherein the primary leukemic cell population is selected from an acute lymphocytic leukemia, an acute myeloid leukemia, a chronic lymphocytic leukemia, a chronic myeloid leukemia and a pre-B acute lymphocytic leukemia.

11. The method of claim 1, wherein the population of human or animal cells is a leukemic cell line selected from bone marrow acute myelogenous leukemia, acute T-cell leukemia, peripheral blood acute lymphoblastic leukemia, chronic myeloid leukemia, acute monocytic leukemia and B-cell leukemia.

12. The method of claim 1, further comprising the steps:
(a) contacting the population of human or animal cells with at least one cell surface marker indicator capable of selectively binding to the at least one differentially distinguishable subpopulation of cells; and
(b) selectively isolating the at least one subpopulation of cells binding the at least one indicator.

13. The method of claim 1, further comprising the step of contacting the population of human or animal cells with a cell cycle indicator, thereby selectively indicating the multi-drug resistance status of a cell.

14. A method of reducing cellular proliferation by inducing cellular apoptosis, comprising the steps:
(a) identifying at least one differentially distinguishable subpopulation of proliferating cells in a population of human or animal cells;
(b) contacting the at least one differentially distinguishable subpopulation of proliferating cells with a Na⁺/H⁺ exchanger inhibitor; and
(c) inducing a state of apoptosis in the proliferating cells.

15. The method of claim 14 further comprising the steps of:
selectively isolating the proliferating cells from the non-proliferating cells; and
returning the population cells to the human or animal after induction of apoptosis in the proliferating cells.

16. The method of claim 14, further comprising the steps of:
(1) identifying an effective apoptosis-inducing amount of a pharmaceutically acceptable Na⁺/H⁺ exchanger inhibitor; and
(2) administering the pharmaceutically acceptable effective apoptosis-inducing amount of a Na⁺/H⁺ exchanger inhibitor to a human or animal.

17. The method of claim 14, further comprising the steps:
(1) contacting the population of human or animal cells with a cell surface antigen indicator capable of selectively binding to a cell surface antigen; and
(2) identifying a subpopulation of proliferating cells having the at least one cell surface marker.

18. The method of claim 14, wherein the population of human or animal cells is derived from a tissue, wherein the tissue is disrupted, thereby yielding a suspension of cells.

19. The method of claim 14, wherein the population of human or animal cells is a peripheral blood cell population, bone marrow cell population, a leukemic cell line population or a primary leukemic cell line population.

20. The method of claim 19, wherein the leukemic cell line is bone marrow acute myelogenous leukemia, acute T-cell leukemia, peripheral blood acute lymphoblastic leukemia, chronic myeloid leukemia, acute monocytic leukemia or B-cell leukemia.

21. The method of claim 19, wherein the primary leukemic cell population is an acute lymphocytic leukemia, an acute myeloid leukemia, a chronic lymphocytic leukemia, a chronic myeloid leukemia or a pre-B acute lymphocytic leukemia.

22. The method of claim 14, wherein the Na⁺/H⁺ exchanger inhibitor is amiloride or an amiloride derivative.

23. The method of claim 22, wherein the amiloride derivative is selected from 5-N,N-hexamethylene)-amiloride (HMA), 5-(N,N-ethyl-N-isopropyl)-amiloride (EIPA), 5-N-methyl-N-isobutyl)-amiloride (MIA), 5-(N-methyl-N-isobutyl)-amiloride (MIBA), simvastatin and phenamil.

24. The method of claim 14, wherein the Na⁺/H⁺ exchanger inhibitor is a non-amiloride.

25. The method of claim 24, wherein the non-amiloride is (2-methyl-5-(methylsulfonyl)-4-pyrrolobenzoyl)-guanidine (EMD), (3-methylsulfonyl-4-piperidinobenzoyl)guanidine methanesulfonate) (Hoe 694), CARIPORIDE™ (Hoe 642), cimetidine, clonidine or hormaline.

26. The method of claim 14, further comprising the steps:
(a) contacting the population of human or animal cells with a pH indicator;
(b) delivering the human or animal cells to a device capable of detecting the pH indicator;
(c) determining the pHᵢ of the human or animal cells from the pH indicator; and
(d) determining the percentage of cells of the population of human or animal cells that are in the S-phase of the cell cycle as a function of the pHᵢ of the cells.

27. The method of claim 26, wherein the pH indicator is a fluorescent pH indicator.

28. The method of claim 26, wherein the fluorescent pH indicator is 1,4-diacetoxy-2,3-dicyanobenzene (ADB), 4-methylumbelliferone (4-MU), 2′,7′-bis-carboxyethyl-5(6)-carboxyfluorescein (BCECF) or carboxy-SemiNapthoRhoda-Fluor-1 acetoxymethyl ester, acetate (carboxy SNARF-1 AM).

29. The method of claim 26, wherein the fluorescent pH indicator is carboxy-SemiNapthoRhoda-Fluor-1 acetoxymethyl ester, acetate (carboxy SNARF-1 AM).

30. A kit for selectively identifying a subpopulation of cells in a population of cells, comprising packaging material, a pH indicator and at least one cell surface antigen indicator capable of selectively binding to a cell surface antigen, and instructions for using the pH indicator and the at least one cell surface antigen indicator for selectively identifying a subpopulation of human or animal cells.

31. The kit of claim 30, further comprising a cell cycle indicator and instructions for the use thereof to determine the cell cycle status of a cell.

32. The kit of claim 30, further comprising at least one enzyme capable of digesting connective tissue and thereby disrupting a tissue.

33. A kit for reducing cellular proliferation in a subpopulation of cells by inducing cellular apoptosis, comprising packaging, at least one pH indicator, at least one cell surface antigen indicator capable of selectively binding to a cell surface marker, at least one Na⁺/H⁺ exchanger inhibitor, and instructions for using the at least one pH indicator, at least one indicator capable of selectively binding to a cell surface marker, and the at least one Na⁺/H⁺ exchanger inhibitor for reducing cellular proliferation by inducing cellular apoptosis.

34. The kit of claim 33, further comprising a cell cycle indicator and instructions for the use thereof to determine the cell cycle status of a cell.

35. The kit of claim 33, further comprising at least one enzyme capable of digesting connective tissue and thereby disrupting a tissue.
